SEQUENCE LISTING (69 sequences)

SEQ ID NO: 1 - protein, 540 aa, Homo sapiens [sequence omitted]

SEQ ID NO: 2 - protein, 118 aa, synthetic construct
  REGION 1..118: note = Consensus
  VARIANT 28: note = X can be any naturally occurring amino acid
  VARIANT 33: note = X can be any naturally occurring amino acid
  VARIANT 50: note = X can be any naturally occurring amino acid
  VARIANT 52..54: note = X can be any naturally occurring amino acid
  VARIANT 57..58: note = X can be any naturally occurring amino acid
  VARIANT 62: note = X can be any naturally occurring amino acid
  VARIANT 65: note = X can be any naturally occurring amino acid
  VARIANT 99..100: note = X can be any naturally occurring amino acid
  VARIANT 102: note = X can be any naturally occurring amino acid
  VARIANT 104..105: note = X can be any naturally occurring amino acid
  VARIANT 107: note = X can be any naturally occurring amino acid
QVQLVQSGAEVKKPGASVKVSCKASGYXFTSYXMHWVRQAPGQGLEWMGXIXXXGGXXNYNXKFXSRVTMTRDTSTSTVYMELSSLRSEDTAVYYCARXXPXYXXDXWGQGTTVTVSS

SEQ ID NO: 3 - protein, 118 aa, synthetic construct
  REGION 1..118: note = Reference
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 4 - protein, 118 aa, synthetic construct
  REGION 1..118: note = AB-1
QVQLVQSGAEVKKPGASVKVSCKASGYTFTSYYMHWVRQAPGQGLEWMGEILPGGGTTNYNQKFKSRVTMTRDTSTSTVYMELSSLRSEDTAVYYCARESPLYASDIWGQGTTVTVSS

SEQ ID NO: 5 - protein, 118 aa, synthetic construct
  REGION 1..118: note = AB-2
QVQLVQSGAEVKKPGASVKVSCKASGYTFTSYYMHWVRQAPGQGLEWMGWIEPFGGRTNYNEKFKSRVTMTRDTSTSTVYMELSSLRSEDTAVYYCARERPLYAMDVWGQGTTVTVSS

SEQ ID NO: 6 - protein, 118 aa, synthetic construct
  REGION 1..118: note = AB-3
QVQLVQSGAEVKKPGASVKVSCKASGYTFTSYWMHWVRQAPGQGLEWMGEIDPSGGSTNYNQKFQSRVTMTRDTSTSTVYMELSSLRSEDTAVYYCARERPLYASDVWGQGTTVTVSS

SEQ ID NO: 7 - protein, 118 aa, synthetic construct
  REGION 1..118: note = AB-4
QVQLVQSGAEVKKPGASVKVSCKASGYTFTSYYMHWVRQAPGQGLEWMGEIRPSGGSTNYNEKFKSRVTMTRDTSTSTVYMELSSLRSEDTAVYYCARDRPLYSMDVWGQGTTVTVSS

SEQ ID NO: 8 - protein, 118 aa, synthetic construct
  REGION 1..118: note = AB-5
QVQLVQSGAEVKKPGASVKVSCKASGYTFTSYWMHWVRQAPGQGLEWMGEISPSGGSTNYNEKFKSRVTMTRDTSTSTVYMELSSLRSEDTAVYYCARERPLYAMDVWGQGTTVTVSS

SEQ ID NO: 9 - protein, 118 aa, synthetic construct
  REGION 1..118: note = AB-6
QVQLVQSGAEVKKPGASVKVSCKASGYTFTSYYMHWVRQAPGQGLEWMGEISPFGGTTNYNEKFKSRVTMTRDTSTSTVYMELSSLRSEDTAVYYCARERPLYSSDVWGQGTTVTVSS

SEQ ID NO: 10 - protein, 118 aa, synthetic construct
  REGION 1..118: note = AB-7
QVQLVQSGAEVKKPGASVKVSCKASGYTFTSYYMHWVRQAPGQGLEWMGEIFPSGGRTNYNEKFKSRVTMTRDTSTSTVYMELSSLRSEDTAVYYCARERPFYASDLWGQGTTVTVSS

SEQ ID NO: 11 - protein, 118 aa, synthetic construct
  REGION 1..118: note = AB-8
QVQLVQSGAEVKKPGASVKVSCKASGYTFTSYYMHWVRQAPGQGLEWMGEILPSGGRTNYNEKFKSRVTMTRDTSTSTVYMELSSLRSEDTAVYYCARERPLYAMDIWGQGTTVTVSS

SEQ ID NO: 12 - protein, 118 aa, synthetic construct
  REGION 1..118: note = AB-9
QVQLVQSGAEVKKPGASVKVSCKASGYTFTSYYMHWVRQAPGQGLEWMGEIKPSGGTSNYNEKFKSRVTMTRDTSTSTVYMELSSLRSEDTAVYYCARDRPLYYSDVWGQGTTVTVSS

SEQ ID NO: 13 - protein, 118 aa, synthetic construct
  REGION 1..118: note = AB-10
QVQLVQSGAEVKKPGASVKVSCKASGYTFTSYYMHWVRQAPGQGLEWMGEILPSGGRTNYNEKFKSRVTMTRDTSTSTVYMELSSLRSEDTAVYYCARERPLYAMDVWGQGTTVTVSS

SEQ ID NO: 14 - protein, 118 aa, synthetic construct
  REGION 1..118: note = AB-11
QVQLVQSGAEVKKPGASVKVSCKASGYSFTSYYMHWVRQAPGQGLEWMGEIDLSGGSTNYNQKFQSRVTMTRDTSTSTVYMELSSLRSEDTAVYYCARERPLYAFDVWGQGTTVTVSS

SEQ ID NO: 15 - protein, 107 aa, synthetic construct
  REGION 1..107: note = Consensus
  VARIANT 31..32: note = X can be any naturally occurring amino acid
  VARIANT 53..54: note = X can be any naturally occurring amino acid
  VARIANT 56: note = X can be any naturally occurring amino acid
  VARIANT 91: note = X can be any naturally occurring amino acid
  VARIANT 94..96: note = X can be any naturally occurring amino acid
DIQMTQSPSSLSASVGDRVTITCRASQGISXXLAWYQQKPGKAPKLLIYSASXXYXGVPSRFSGSGSGTDFTFTISSLQPEDIATYYCQQXYSXXXTFGQGTKLEIK

SEQ ID NO: 16 - protein, 107 aa, synthetic construct
  REGION 1..107: note = Reference, AB-3, 5, 11
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 17 - protein, 107 aa, synthetic construct
  REGION 1..107: note = AB-1
DIQMTQSPSSLSASVGDRVTITCRASQGISTALAWYQQKPGKAPKLLIYSASYLYTGVPSRFSGSGSGTDFTFTISSLQPEDIATYYCQQRYSLWRTFGQGTKLEIK

SEQ ID NO: 18 - protein, 107 aa, synthetic construct
  REGION 1..107: note = AB-2
DIQMTQSPSSLSASVGDRVTITCRASQGISGALAWYQQKPGKAPKLLIYSASYRYTGVPSRFSGSGSGTDFTFTISSLQPEDIATYYCQQRYSLSRTFGQGTKLEIK

SEQ ID NO: 19 - protein, 107 aa, synthetic construct
  REGION 1..107: note = AB-4
DIQMTQSPSSLSASVGDRVTITCRASQGISSALAWYQQKPGKAPKLLIYSASYRYTGVPSRFSGSGSGTDFTFTISSLQPEDIATYYCQQHYSSSWTFGQGTKLEIK

SEQ ID NO: 20 - protein, 107 aa, synthetic construct
  REGION 1..107: note = AB-6
DIQMTQSPSSLSASVGDRVTITCRASQGISSALAWYQQKPGKAPKLLIYSASYLYSGVPSRFSGSGSGTDFTFTISSLQPEDIATYYCQQRYSLWYTFGQGTKLEIK

SEQ ID NO: 21 - protein, 107 aa, synthetic construct
  REGION 1..107: note = AB-7
DIQMTQSPSSLSASVGDRVTITCRASQGISSALAWYQQKPGKAPKLLIYSASNLYTGVPSRFSGSGSGTDFTFTISSLQPEDIATYYCQQRYSLWRTFGQGTKLEIK

SEQ ID NO: 22 - protein, 107 aa, synthetic construct
  REGION 1..107: note = AB-8
DIQMTQSPSSLSASVGDRVTITCRASQGISSALAWYQQKPGKAPKLLIYSASYRYTGVPSRFSGSGSGTDFTFTISSLQPEDIATYYCQQRYSTWRTFGQGTKLEIK

SEQ ID NO: 23 - protein, 107 aa, synthetic construct
  REGION 1..107: note = AB-9
DIQMTQSPSSLSASVGDRVTITCRASQGISGSLAWYQQKPGKAPKLLIYSASYRYTGVPSRFSGSGSGTDFTFTISSLQPEDIATYYCQQYYSNYWTFGQGTKLEIK

SEQ ID NO: 24 - protein, 107 aa, synthetic construct
  REGION 1..107: note = AB-10
DIQMTQSPSSLSASVGDRVTITCRASQGISSALAWYQQKPGKAPKLLIYSASYRYTGVPSRFSGSGSGTDFTFTISSLQPEDIATYYCQQRYSVALTFGQGTKLEIK

SEQ ID NO: 25 - protein, 8 aa, synthetic construct
  REGION 1..8: note = HCDR1; Consensus
  VARIANT 8: note = X can be any naturally occurring amino acid
GYTFTSYX

SEQ ID NO: 26 - protein, 8 aa, synthetic construct
  REGION 1..8: note = HCDR1; Reference, AB-1, 2, 4, 6-10
GYTFTSYY

SEQ ID NO: 27 - protein, 8 aa, synthetic construct
  REGION 1..8: note = HCDR1; AB-3, 5
GYTFTSYW

SEQ ID NO: 28 - protein, 8 aa, synthetic construct
  REGION 1..8: note = HCDR1; AB-11
GYSFTSYY

SEQ ID NO: 29 - protein, 8 aa, synthetic construct
  REGION 1..8: note = HCDR2; Consensus
  VARIANT 2: note = X can be any naturally occurring amino acid
  VARIANT 4: note = X can be any naturally occurring amino acid
  VARIANT 7..8: note = X can be any naturally occurring amino acid
IXPXGGXX

SEQ ID NO: 30 - protein, 8 aa, synthetic construct
  REGION 1..8: note = HCDR2; Reference
ISPFGGRT

SEQ ID NO: 31 - protein, 8 aa, synthetic construct
  REGION 1..8: note = HCDR2; AB-1
ILPGGGTT

SEQ ID NO: 32 - protein, 8 aa, synthetic construct
  REGION 1..8: note = HCDR2; AB-2
IEPFGGRT

SEQ ID NO: 33 - protein, 8 aa, synthetic construct
  REGION 1..8: note = HCDR2; AB-3
IDPSGGST

SEQ ID NO: 34 - protein, 8 aa, synthetic construct
  REGION 1..8: note = HCDR2; AB-4
IRPSGGST

SEQ ID NO: 35 - protein, 8 aa, synthetic construct
  REGION 1..8: note = HCDR2; AB-5
ISPSGGST

SEQ ID NO: 36 - protein, 8 aa, synthetic construct
  REGION 1..8: note = HCDR2; AB-6
ISPFGGTT

SEQ ID NO: 37 - protein, 8 aa, synthetic construct
  REGION 1..8: note = HCDR2; AB-7
IFPSGGRT

SEQ ID NO: 38 - protein, 8 aa, synthetic construct
  REGION 1..8: note = HCDR2; AB-8, 10
ILPSGGRT

SEQ ID NO: 39 - protein, 8 aa, synthetic construct
  REGION 1..8: note = HCDR2; AB-9
IKPSGGTS

SEQ ID NO: 40 - protein, 8 aa, synthetic construct
  REGION 1..8: note = HCDR2; AB-11
IDLSGGST

SEQ ID NO: 41 - protein, 11 aa, synthetic construct
  REGION 1..11: note = HCDR3; Consensus
  VARIANT 3..4: note = X can be any naturally occurring amino acid
  VARIANT 6: note = X can be any naturally occurring amino acid
  VARIANT 8..9: note = X can be any naturally occurring amino acid
  VARIANT 11: note = X can be any naturally occurring amino acid
ARXXPXYXXDX

SEQ ID NO: 42 - protein, 11 aa, synthetic construct
  REGION 1..11: note = HCDR3; Reference
ARERPLYASDL

SEQ ID NO: 43 - protein, 11 aa, synthetic construct
  REGION 1..11: note = HCDR3; AB-1
ARESPLYASDI

SEQ ID NO: 44 - protein, 11 aa, synthetic construct
  REGION 1..11: note = HCDR3; AB-2, 5, 10
ARERPLYAMDV

SEQ ID NO: 45 - protein, 11 aa, synthetic construct
  REGION 1..11: note = HCDR3; AB-3
ARERPLYASDV

SEQ ID NO: 46 - protein, 11 aa, synthetic construct
  REGION 1..11: note = HCDR3; AB-4
ARDRPLYSMDV

SEQ ID NO: 47 - protein, 11 aa, synthetic construct
  REGION 1..11: note = HCDR3; AB-6
ARERPLYSSDV

SEQ ID NO: 48 - protein, 11 aa, synthetic construct
  REGION 1..11: note = HCDR3; AB-7
ARERPFYASDL

SEQ ID NO: 49 - protein, 11 aa, synthetic construct
  REGION 1..11: note = HCDR3; AB-8
ARERPLYAMDI

SEQ ID NO: 50 - protein, 11 aa, synthetic construct
  REGION 1..11: note = HCDR3; AB-9
ARDRPLYYSDV

SEQ ID NO: 51 - protein, 11 aa, synthetic construct
  REGION 1..11: note = HCDR3; AB-11
ARERPLYAFDV

SEQ ID NO: 52 - protein, 6 aa, synthetic construct
  REGION 1..6: note = LCDR1; Consensus
  VARIANT 5..6: note = X can be any naturally occurring amino acid
QGISXX

SEQ ID NO: 53 - protein, 6 aa, synthetic construct
  REGION 1..6: note = LCDR1; Reference, AB-3-8, 10, 11
QGISSA

SEQ ID NO: 54 - protein, 6 aa, synthetic construct
  REGION 1..6: note = LCDR1; AB-1
QGISTA

SEQ ID NO: 55 - protein, 6 aa, synthetic construct
  REGION 1..6: note = LCDR1; AB-9
QGISGS

SEQ ID NO: 56 - skipped

SEQ ID NO: 57 - protein, 9 aa, synthetic construct
  REGION 1..9: note = LCDR3; Consensus
  VARIANT 3: note = X can be any naturally occurring amino acid
  VARIANT 6..8: note = X can be any naturally occurring amino acid
QQXYSXXXT

SEQ ID NO: 58 - protein, 9 aa, synthetic construct
  REGION 1..9: note = LCDR3; Reference, AB-1, 3, 5, 7, 11
QQRYSLWRT

SEQ ID NO: 59 - protein, 9 aa, synthetic construct
  REGION 1..9: note = LCDR3; AB-2
QQRYSLSRT

SEQ ID NO: 60 - protein, 9 aa, synthetic construct
  REGION 1..9: note = LCDR3; AB-4
QQHYSSSWT

SEQ ID NO: 61 - protein, 9 aa, synthetic construct
  REGION 1..9: note = LCDR3; AB-6
QQRYSLWYT

SEQ ID NO: 62 - protein, 9 aa, synthetic construct
  REGION 1..9: note = LCDR3; AB-8
QQRYSTWRT

SEQ ID NO: 63 - protein, 9 aa, synthetic construct
  REGION 1..9: note = LCDR3; AB-9
QQYYSNYWT

SEQ ID NO: 64 - protein, 9 aa, synthetic construct
  REGION 1..9: note = LCDR3; AB-10
QQRYSVALT

SEQ ID NO: 65 - protein, 330 aa, synthetic construct [sequence omitted]

SEQ ID NO: 66 - protein, 327 aa, synthetic construct
  REGION 1..327: note = antibody heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 107 aa, synthetic construct [sequence omitted]

SEQ ID NO: 68 - protein, 106 aa, synthetic construct
  REGION 1..106: note = antibody light chain constant region
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 69 - protein, 6 aa, synthetic construct
  REGION 1..6: note = LCDR1: AB-2
QGISGA

(19) United States
(12) Patent Application Publication
(10) Pub. No.: US 2025/0368753 A1
(43) Pub. Date: Dec. 4, 2025

(54) MODULATORS OF PROPROTEIN CONVERTASE SUBTILISIN/KEXIN TYPE 9 (PCSK9)

(71) Applicant: Flagship Pioneering Innovations VI, LLC, Cambridge, MA (US)

(72) Inventors: Daniel Solomon Leventhal, Medford, MA (US); Brinda Monian, Medford, MA (US); Jenhan Tao, Boston, MA (US)

(21) Appl. No.: 18/703,246

(22) PCT Filed: Oct. 21, 2022
(86) PCT No.: PCT/US2022/078543

§ 371 Date: Apr. 19, 2024

Related U.S. Application Data
(60) Provisional application No. 63/270,484, filed on Oct. 21, 2021.

(51) Int. Cl. C07K 16/40

(52) U.S. Cl. CPC C07K 16/40; C07K 2317/76; C07K 2317/92

(57) ABSTRACT

The disclosure provides, in various embodiments, polypeptides that specifically bind to proprotein convertase subtilisin/kexin type 9 (PCSK9) (e.g., human PCSK9). The disclosure also provides, in various embodiments, methods for treating diseases associated with increased low-density lipoprotein (LDL) levels in the blood (e.g., hypercholesterolemia and its related cardiovascular disease risk).

### RELATED APPLICATION(S)

This application is the U.S. National Stage of International Application No. PCT/US2022/078543, filed Oct. 21, 2022, which designates the U.S., published in English, and claims the benefit of U.S. Provisional Application No. 63/270,484, filed on Oct. 21, 2021. The entire teachings of the above applications are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) 57081052002-SL.xml; created Mar. 10, 2025, 99,637 Bytes in size.

### BACKGROUND

Proprotein convertase subtilisin/kexin type 9 (PCSK9), a member of the proprotein convertase family of enzymes encoded by the PCSK9 gene in humans, plays an important role in the regulation of cholesterol homeostasis (Bergeron et al., Circulation 132(17):1648-66 (2015)). PCSK9 reduces low-density lipoprotein (LDL) uptake by binding to hepatic LDL receptors and promoting their lysosomal degradation (Id.). Gain-of-function mutations in PCSK9 have been associated with premature cardiovascular disease, whereas the more commonly expressed loss-of-function mutations in PCSK9 are associated with reduced cardiovascular disease risk (Id.). Because inhibiting PCSK9 is expected to provide therapeutic benefits in managing/treating hypercholesterolemia and its related cardiovascular diseases, there is a critical need to develop novel polypeptides that specifically bind and inhibit PCSK9.

### SUMMARY

The disclosure generally relates to compositions (e.g., polypeptides, polynucleotides, vectors, host cells, pharmaceutical compositions) and methods that are useful for inhibiting (e.g., blocking) PCSK9 activity (e.g., by binding to hepatic LDL receptors and promoting their lysosomal degradation).

In some embodiments, the disclosure provides a polypeptide that specifically binds a PCSK9, wherein the polypeptide comprises:
a) an immunoglobulin V_{H} comprising an amino acid sequence that is at least 55% identical to SEQ ID NO:3;
b) an immunoglobulin V_{L} comprising an amino acid sequence that is at least 55% identical to SEQ ID NO:16; or
c) both a) and b),
wherein the polypeptide does not comprise all five of SEQ ID NO:26, SEQ ID NO:30, SEQ ID NO: 42, SEQ ID NO:53, and SEQ ID NO:58.

In some embodiments, the disclosure provides a polypeptide that specifically binds a PCSK9, wherein the polypeptide comprises:
a) an immunoglobulin V_{H} comprising an amino acid sequence that is at least 55% identical to SEQ ID NO:3, wherein the V_{H} does not comprise SEQ ID NO:26, SEQ ID NO:30, SEQ ID NO:42, or a combination thereof;
b) an immunoglobulin V_{L} comprising an amino acid sequence that is at least 55% identical to SEQ ID NO:15, wherein the V_{L} does not comprise SEQ ID NO:53, and SEQ ID NO:58, or a combination thereof; or
c) both a) and b).

In some embodiments, the disclosure provides a polypeptide that specifically binds a PCSK9, comprising a paratope that is identical to the paratope of an antibody comprising an immunoglobulin V_{H} and an immunoglobulin V_{L}, wherein the V_{H} and V_{L} of the antibody are selected from the following V_{H}/V_{L} combinations:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); and
SEQ ID NO:14/SEQ ID NO:16 (AB-11).

In some embodiments, the disclosure provides a polypeptide that specifically binds a PCSK9, wherein:
a) the polypeptide comprises:
   i. an immunoglobulin V_{H} comprising a HCDR1, a HCDR2 and a HCDR3 that are identical to the HCDR1, HCDR2 and HCDR3, respectively, of any one of SEQ ID NOs: 4-14; and
   ii. an immunoglobulin V_{L} comprising a LCDR1, a LCDR2 and a LCDR3 that are identical to the LCDR1, LCDR2 and LCDR3, respectively, of any one of SEQ ID NOs: 16-24; or
b) the polypeptide comprises:
   i. an immunoglobulin V_{H} comprising a HCDR1, a HCDR2 and a HCDR3 that are identical to the HCDR1, HCDR2 and HCDR3, respectively, of SEQ ID NO:3; and
ii. an immunoglobulin V_{L} comprising a LCDR1, a LCDR2 and a LCDR3 that are identical to the LCDR1, LCDR2 and LCDR3, respectively, of any one of SEQ ID NOs: 17-24.

In some embodiments, the disclosure provides a polypeptide that specifically binds a PCSK9, wherein the polypeptide comprises a V_{H} comprising SEQ ID NO:2, a V_{L} comprising SEQ ID NO:15, or both, wherein:
X₁ is not T;
X₂ is not Y;
X₃ is not E;
X₄ is not S;
X₅ is not P;
X₆ is not F;
X₇ is not R;
X₈ is not T;
X₉ is not E;
X₁₀ is not K;
X₁₁ is not E;
X₁₂ is not R;
X₁₃ is not L;
X₁₄ is not A;
X₁₅ is not S;
X₁₆ is not L;
X₁₇ is not S;
X₁₈ is not A;
X₁₉ is not Y;
X₂₀ is not R;
X₂₁ is not T;
X₂₂ is not R;
X₂₃ is not L;
X₂₄ is not W; or
X₂₅ is not R, or
any combination of the foregoing.

In some embodiments, the disclosure provides a fusion protein comprising one or more of the polypeptides described herein.

In some embodiments, the disclosure provides one or more polynucleotides encoding any one of the polypeptides or fusion proteins described herein.

In some embodiments, the disclosure provides an expression vector comprising any one or more of the polynucleotides described herein.

In some embodiments, the disclosure provides an expression host cell comprising any one or more of the polynucleotides or expression vectors described herein.

In some embodiments, the disclosure provides a composition comprising any one or more of the polypeptides or fusion proteins described herein. In some embodiments, the composition is a pharmaceutical composition.

In some embodiments, the disclosure provides a method of treating a subject in need thereof, comprising administering to the subject an effective amount of any one of the compositions (e.g., pharmaceutical compositions) described herein.

In some embodiments, the disclosure provides a method of treating a disease or condition associated with increased low-density lipoprotein (LDL) level in the blood in a subject in need thereof, comprising administering to the subject an effective amount of any one of the compositions (e.g., pharmaceutical compositions) described herein.

In some embodiments, the disclosure provides a method of preventing a disease or condition associated with an increased LDL level in the blood in a subject in need thereof, comprising administering to the subject an effective amount of any one of the compositions (e.g., pharmaceutical compositions) described herein.

In some embodiments, the disclosure provides a method of reducing circulating LDL in a subject in need thereof, comprising administering to the subject an effective amount of any one of the compositions (e.g., pharmaceutical compositions) described herein.

In some embodiments, the disclosure provides a method of modulating (e.g., decreasing, increasing) PCSK9 activity in a subject in need thereof, comprising administering to the subject an effective amount of any one of the compositions (e.g., pharmaceutical compositions) described herein.

In some embodiments, the disclosure provides a method of modulating (e.g., decreasing, increasing) PCSK9 activity in a mammalian cell, comprising contacting the cell with any one of the polypeptides, fusion proteins or compositions described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 depicts the amino acid sequence of human proprotein convertase subtilisin/kexin type 9 (PCSK9; SEQ ID NO:1). “*” indicates the epitope residues bound by the Reference Antibody disclosed herein and certain PCSK9 binders of the disclosure.

FIGS. 2A-2C depict alignments of non-limiting examples of heavy chain complementarity determining region (HCDR) amino acid sequences. HCDR sequences are indicated using bold letters. “*” indicates paratope residues. Shading indicates non-limiting examples of variable residues (designated throughout this disclosure by “Xₙ”) in the depicted sequences. The location of variable positions “Xₙ,” where n is a number from 1, 2, 4-8 and 11-16, is indicated by the corresponding number at the bottom of the alignments.

FIGS. 3A-3C depict alignments of non-limiting examples of light chain complementarity determining region (LCDR) amino acid sequences. LCDR sequences are indicated using bold letters. “*” indicates paratope residues. Shading indicates non-limiting examples of variable residues (designated throughout this disclosure by “Xₙ”) in the depicted sequences. The location of variable positions “Xₙ,” where n is a number from 17, 18, 22-25, is indicated by the corresponding number at the bottom of the alignments.

FIG. 4A depicts a non-limiting example of a heavy chain variable domain of a PCSK9-binding polypeptide. HCDR sequences are indicated using bold and underlined letters. “*” indicates paratope residues. The location of variable positions “Xₙ,” where n is a number from 1-16, is indicated by the corresponding number at the bottom of the alignments.

FIG. 4B depicts a non-limiting example of a light chain variable domain of a PCSK9-binding polypeptide. LCDR sequences are indicated using bold and underlined letters. “*” indicates paratope residues. The location of variable positions “Xₙ,” where n is a number from 17-25, is indicated by the corresponding number at the bottom of the alignments.

FIG. 5A shows the total number of sequences at each mutational distance (from 2 to 14 amino acid mutations from reference sequence or native) for the entire antibody variant set. Each bar is broken up into the various groups representing different computational approaches to sequence design.

FIG. 5B depicts the computationally predicted stealth score compared to the predicted developability score for each antibody variant in the entire variant set with the sequence design methodology utilized to generate that sequence noted. The predicted stealth score refers to the probability of peptides derived from the variant to be presented on MHC II molecules and serves as a proxy for predicted immunogenicity. The predicted developability score refers to the predicted self-association measurement which would be measured by AC-SINS assay. The red dotted line shows the prediction calculated for the Reference Antibody or native sequence.

FIG. 6 depicts the self-association rate of antibody variants as measured by AC-SINS assay. Each dot depicts an individual variant, with variants grouped by whether the computational design methodology specifically targeted optimization of the AC-SINS parameter or not. The measured AC-SINS value for the Reference Antibody is depicted by the top labeled dotted line and the targeted acceptable AC-SINS threshold of 10 is shown as the bottom labeled dotted line. The percentage of variants demonstrating improvements in AC-SINS compared to Reference Antibody are noted above each variant set grouping.

FIG. 7 depicts the results of a Dendritic Cell (DC) internalization assay with individual dots representing results obtained for the noted antibody with DCs derived from an individual Peripheral Blood Mononuclear Cell (PBMC) donor. The average and standard deviations are shown per antibody, with the horizonal dotted line noting the average DC internalization observed for the Reference Antibody. A students-t test was performed to determine statistical significance between the results observed for each antibody compared to Reference Antibody (**=p<0.005, ***=p<0.0005). A control is shown as a low immunogenicity reference control.

FIG. 8 depicts the results of an in vitro T cell proliferation assay with each bar denoting the percentage of donors (out of 42 individual donors evaluated) responding to the indicated test article or benchmark control. Each donor and condition were tested as six biological replicates, with the average percentage of proliferated T cells for a specific test article compared to the average proliferation observed in the background control on a per donor basis. If a donor exhibited a two-fold or higher levels of proliferation compared to the background, it was considered to be a positive responder. High immunogenicity benchmark controls (Alemtuzumab and Tdap vaccine) and low immunogenicity benchmark controls (Secukinumab, Evolocumab and Alirocumab) were evaluated in parallel to demonstrate relative percentage responding donors for those clinically evaluated molecules.

FIG. 9 depicts the results of an MHC-II associated peptide proteomics (MAPPs) assay. Results from the three proteins evaluated (Reference, AB-11 and AB-8) with unique peptides detected from ten independent donors are shown. Each unique peptide is mapped back to the protein sequence from which it was derived (indicated on the x-axis as residue index and divided into heavy and light chain sequences). The sum total number of unique peptides detected among all ten donors is noted for each protein.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The disclosure is not limited by the illustrated ordering of acts or events, as some acts may occur in different orders and/or concurrently with other acts or events. Furthermore, not all illustrated acts, steps or events are required to implement a methodology in accordance with the disclosure. Many of the techniques and procedures described, or referenced herein, are well understood and commonly employed using conventional methodology by those skilled in the art.

### PCSK9-Binding Polypeptides of the Disclosure

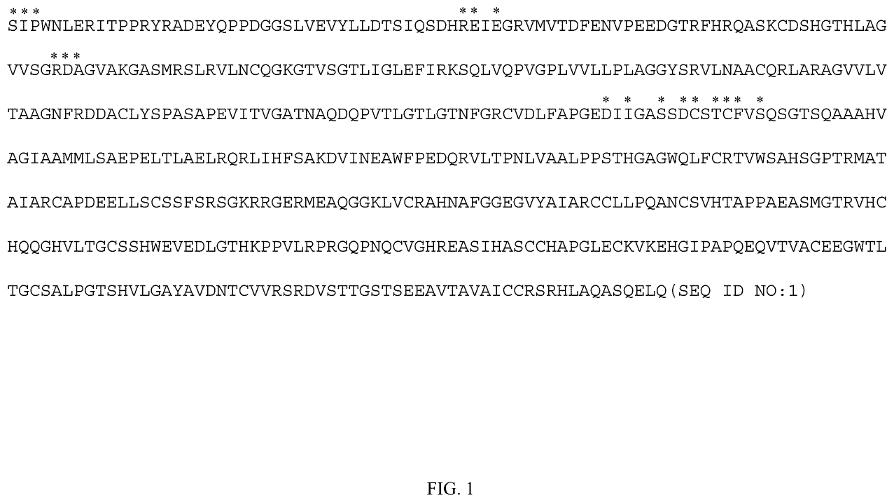

The present disclosure generally relates to polypeptides that bind to proprotein convertase subtilisin/kexin type 9 (PCSK9) protein(s), and uses thereof. Non-limiting examples of PCSK9 protein sequences include human PCSK9 (e.g., UniProt ID: Q8NBP7; NCBI PDB: 2P4E_P). The sequence identified as SEQ ID NO: 1 below is the sequence of a truncated active form of human PCSK9 (also shown in FIG. 1).

(SEQ ID NO: 1)
SIPWNLERITPPRYRADEYQPPDGGSLVEVYLLDTSIQSDHREIEGRVMV
TDFENVPEEDGTRFHRQASKCDSHGTHLAGVVSGRDAGVAKGASMRSLRV
LNCQGKGTVSGTLIGLEFIRKSQLVQPVGPLVVLLPLAGGYSRVLNAACQ
RLARAGVVLVTAAGNFRDDACLYSPASAPEVITVGATNAQDQPVTLGTLG
TNFGRCVDLFAPGEDIIGASSDCSTCFVSQSGTSQAAAHVAGIAAMMLSA
EPELTLAELRQRLIHFSAKDVINEAWFPEDQRVLTPNLVAALPPSTHGAG
WQLFCRTVWSAHSGPTRMATAIARCAPDEELLSCSSFSRSGKRRGERMEA
QGGKLVCRAHNAFGGEGVYAIARCCLLPQANCSVHTAPPAEASMGTRVHC
HQQGHVLTGCSSHWEVEDLGTHKPPVLRPRGQPNQCVGHREASIHASCCH
APGLECKVKEHGIPAPQEQVTVACEEGWTLTGCSALPGTSHVLGAYAVDN
TCVVRSRDVSTTGSTSEEAVTAVAICCRSRHLAQASQELQ

In some embodiments, the disclosure provides a polypeptide that binds to a PCSK9 (e.g., SEQ ID NO:1 at an epitope depicted in FIG. 1) and comprises an immunoglobulin light chain variable region, an immunoglobulin heavy chain variable region, or an immunoglobulin light chain variable region and an immunoglobulin heavy chain variable region, wherein the polypeptide does not comprise all five of SEQ ID NO:26, SEQ ID NO:30, SEQ ID NO:42, SEQ ID NO: 53, and SEQ ID NO:58. In some embodiments, the polypeptide comprises 1, 2, 3 or 4 CDRs selected from SEQ ID NO:26, SEQ ID NO:30, SEQ ID NO:42, SEQ ID NO:53, and SEQ ID NO: 58.

In some embodiments, the disclosure provides a polypeptide that specifically binds a PCSK9, wherein the polypeptide comprises:
a) an immunoglobulin heavy chain variable region (V_{H}) comprising an amino acid sequence that is at least 55% (e.g., at least 60, 65, 70, 75, 80, 85, 90, 95, 98, or 99%) identical to SEQ ID NO:3;
b) an immunoglobulin light chain variable region (V_{L}) comprising an amino acid sequence that is at least 55% (e.g., at least 60, 65, 70, 75, 80, 85, 90, 95, 98, or 99%) identical to SEQ ID NO:16; or
c) both a) and b),
wherein the polypeptide does not comprise all five of SEQ ID NO:26, SEQ ID NO:30, SEQ ID NO: 42, SEQ ID NO:53, and SEQ ID NO:58. In some embodiments, the polypeptide comprises 1, 2, 3 or 4 CDRs selected from SEQ ID NO:26, SEQ ID NO:30, SEQ ID NO:42, SEQ ID NO: 53, and SEQ ID NO:58.

In some embodiments, the polypeptide binds to a wildtype PCSK9 protein (e.g., SEQ ID NO: 1). In some embodiments, the polypeptide binds to one or more epitope residues of a wildtype PCSK9 protein (e.g., one or more epitope residues indicated in FIG. 1).

In some embodiments, the polypeptide binds to a variant of PCSK9 comprising one or more amino acid substitutions, deletions and/or insertions relative to a wild type or canonical PCSK9 (e.g., relative to SEQ ID NO:1). In some embodiments, the PCSK9 variant comprises an amino acid sequence that has at least about 90% sequence identity to the wildtype PCSK9 sequence (e.g., SEQ ID NO:1), for example, at least about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the wildtype PCSK9 sequence. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%.

In some embodiments, the polypeptide binds to one or more (e.g., 1, 2, 3, 4, 5, 6, 7, 8, 9, 10, 11, 12, 13, 14, 15, 16, 17 or all 18) human PCSK9 epitope residues selected from S1, I2, P3, R42, E43, E45, R85, D86, A87, D215, I217, S220, D222, C223, T225, C226, F227 and S229 of SEQ ID NO:1.

As used herein, the term “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

The term “polypeptide” “peptide” or “protein” denotes a polymer of at least two amino acids covalently linked by an amide bond, regardless of length or post-translational modification (e.g., glycosylation or phosphorylation). A protein, peptide or polypeptide can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a protein, peptide or polypeptide can also be derivatized (e.g., alkylated) or labeled (e.g., with a detectable label, such as a fluorogen or a hapten) using methods known in the art. A protein, peptide or polypeptide can comprise one or more modifications (e.g., amino acid linkers, acylation, acetylation, amidation, methylation, terminal modifiers (e.g., cyclizing modifications), N-methyl-α-amino group substitution), if desired. In addition, a protein, peptide or polypeptide can be an analog of a known and/or naturally-occurring peptide, for example, a peptide analog having conservative amino acid residue substitution(s).

The term “specifically binding” or “specifically binds” refers to preferential interaction, i.e., significantly higher binding affinity, between an antibody, or an antigen-binding fragment thereof, and its epitope relative to other antigens or amino acid sequences.

As used herein, the term “reference” or “reference polypeptide” refers to a polypeptide (e.g., immunoglobulin molecule) that specifically binds to PCSK9, but is not a polypeptide of the disclosure. The sequence of a reference polypeptide and a polypeptide of the disclosure may be compared to illustrate structural differences between them (e.g., differences at one or more amino acid positions, such as amino acid substitutions). In some embodiments, a polypeptide of the disclosure will have more than insubstantial differences (e.g., one or more substantial differences) in comparison to a reference polypeptide, such that, generally, polypeptides of the disclosure will, under controlled conditions, exhibit one or more (i.e., one, two, or all three) of: a different function, in a different way, to achieve a different result, in comparison to a reference polypeptide. Reference polypeptides will vary by one or more amino acids from a polypeptide of the disclosure, e.g., in some embodiments by 1, 2, 3, 4, 5, 6, 7, 8, 9, 10, 11, 12, 13, 14, 15, 16, 17, 18, 19, 20 or more amino acids. In some embodiments, a reference polypeptide diverges from a polypeptide provided herein by at least about: 0.4, 0.8, 1, 2, 3, 4, 5, 6, 7, 8, 9, 10, 15, 20, 25, 30, 35, 40, 45, 50, 55% or more amino acid identity.

In some embodiments, a reference polypeptide comprises the heavy chain complementarity determining region 1 (HCDR1), heavy chain complementarity determining region 2 (HCDR2) and heavy chain complementarity determining region 3 (HCDR3) amino acid sequences of SEQ ID NO:3, corresponding to SEQ ID NO:26, SEQ ID NO:30, SEQ ID NO:42, respectively, and the light chain complementarity determining region 1 (LCDR1), light chain complementarity determining region 2 (LCDR2) and light chain complementarity determining region 3 (LCDR3) of SEQ ID NO: 16, corresponding to amino acid sequences of SEQ ID NO:53, SAS and SEQ ID NO:58, respectively. The sequence identified as SEQ ID NO:3 is shown in Table 1, and the sequence identified as SEQ ID NO: 16 is shown in Table 2. The sequences identified as SEQ ID NOs: 26, 30, 42, 53, and 58 are shown in Table 3.

In some embodiments, the reference polypeptide is an antibody, referred to herein as “the Reference Antibody,” which comprises a V_{H} region comprising the amino acid sequence of SEQ ID NO:3 and a V_{L} region comprising the amino acid sequence of SEQ ID NO:16. The Reference Antibody is an IgG4 humanized monoclonal antibody that binds to PCSK9. See, e.g., GenBank: AJM94338.1 and AEX16238.1 for the Reference Antibody heavy and light chain sequences, respectively. For additional information about the Reference Antibody, see, e.g., U.S. Pat. Nos. 8,080,243 and 10,472,424, U.S. patent application Ser. No. 14/232,559, published Jun. 12, 2014 as US20140161821 and Ridker et al., Am Heart J. 178:135-44 (2016), the contents of which are incorporated herein by reference in their entireties.

In some embodiments, the disclosure provides a polypeptide that specifically binds a PCSK9, (e.g., at one or more human PCSK9 epitope residues identified herein), comprising:
a) an immunoglobulin V_{H} comprising an amino acid sequence that is at least 55% identical to SEQ ID NO:3, wherein the V_{L} does not comprise SEQ ID NO:26, SEQ ID NO:30 or SEQ ID NO:42, or a combination thereof;
b) an immunoglobulin V_{L} comprising an amino acid sequence that is at least 55% identical to SEQ ID NO: 16, wherein the V_{L} does not comprise SEQ ID NO:53 or SEQ ID NO:58, or both; or
c) both a) and b).

In some embodiments, the polypeptide comprises a V_{H} that is at least about 60% identical to SEQ ID NO:3. For example, the V_{H} can be at least about: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:3.

In some embodiments, the polypeptide comprises a V_{L} that is at least about 60% identical to SEQ ID NO:16. For example, the V_{L} can be at least about: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:16.

In some embodiments, the polypeptide comprises:
a) a V_{H} that is at least 70% identical to SEQ ID NO:3;
b) a V_{L} that is at least 70% identical to SEQ ID NO:16; or
c) both a) and b).

In some embodiments, the polypeptide comprises a V_{H} that is at least about 70% identical to SEQ ID NO:3. For example, the V_{H} can be at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:3. In some embodiments, the V_{H} is at least about 80%, at least about 85%, at least about 90% or at least about 95% identical to SEQ ID NO:3.

In some embodiments, the polypeptide comprises a V_{L} that is at least about 70% identical to SEQ ID NO: 16 For example, the V_{L} can be at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:16. In some embodiments, the V_{L} is at least about 80%, at least about 85%, at least about 90% or at least about 95% identical to SEQ ID NO:16.

In some embodiments, the polypeptide comprises:
a) a V_{H} that is at least about 1% different from SEQ ID NO:3;
b) a V_{L} that is at least about 1% different from SEQ ID NO:16; or
c) both a) and b).

In some embodiments, the polypeptide comprises a V_{H} that is at least about 1% different from SEQ ID NO:3. For example, the V_{H} can be at least about: 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% different from SEQ ID NO:3. In some embodiments, the V_{H} is at least about 5%, at least about 10%, at least about 15%, at least about 20%, at least about 25% or at least about 30% different from SEQ ID NO:3.

In some embodiments, the polypeptide comprises a V_{L} that is at least about 1% different from SEQ ID NO:16. For example, the V_{L} can be at least about: 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% different from SEQ ID NO:16. In some embodiments, the V_{L} is at least about 5%, at least about 10%, at least about 15%, at least about 20%, at least about 25% or at least about 30% different from SEQ ID NO:16.

In some embodiments, the polypeptide comprises:
a) a V_{H} comprising one or more amino acid substitutions relative to SEQ ID NO:3;
b) a V_{L} comprising one or more amino acid substitutions relative to SEQ ID NO:16; or
c) both a) and b).

In some embodiments, the polypeptide comprises a V_{H} comprising one or more amino acid substitutions relative to SEQ ID NO:3. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the V_{H} comprises about 1-20, 1-15, 1-10 or 1-5 amino acid substitutions, relative to SEQ ID NO:3.

In some embodiments, the polypeptide comprises a V_{L} comprising one or more amino acid substitutions relative to SEQ ID NO: 16. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the VI comprises about 1-20, 1-15, 1-10 or 1-5 amino acid substitutions, relative to SEQ ID NO:16.

In some embodiments, the one or more amino acid substitutions are conservative substitutions. The term “conservative amino acid substitution(s)” or “conservative substitution(s)” refers to an amino acid substitution having a value of 0 or greater in BLOSUM62.

In some embodiments, the one or more amino acid substitutions are highly conservative substitutions. The term “highly conservative amino acid substitution(s)” or “highly conservative substitution(s)” refers to an amino acid substitution having a value of at least 1 (e.g., at least 2) in BLOSUM62.

In some embodiments, the polypeptide comprises a paratope that is identical to the paratope of an antibody comprising a V_{H} and a V_{L}, wherein the V_{H} and V_{L} of the antibody are selected from the following V_{H}/V_{L} combinations:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); and
SEQ ID NO:14/SEQ ID NO:16 (AB-11).

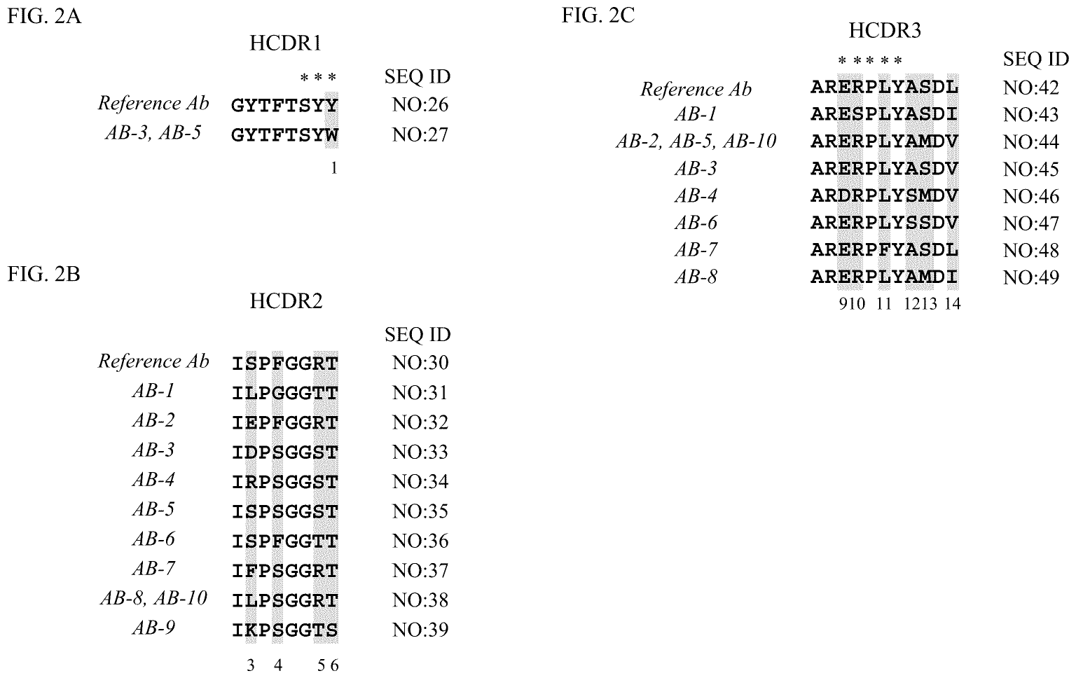

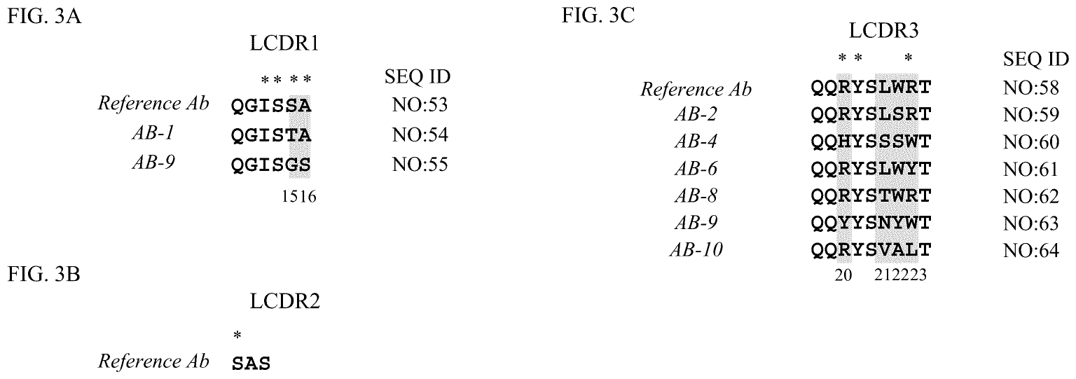

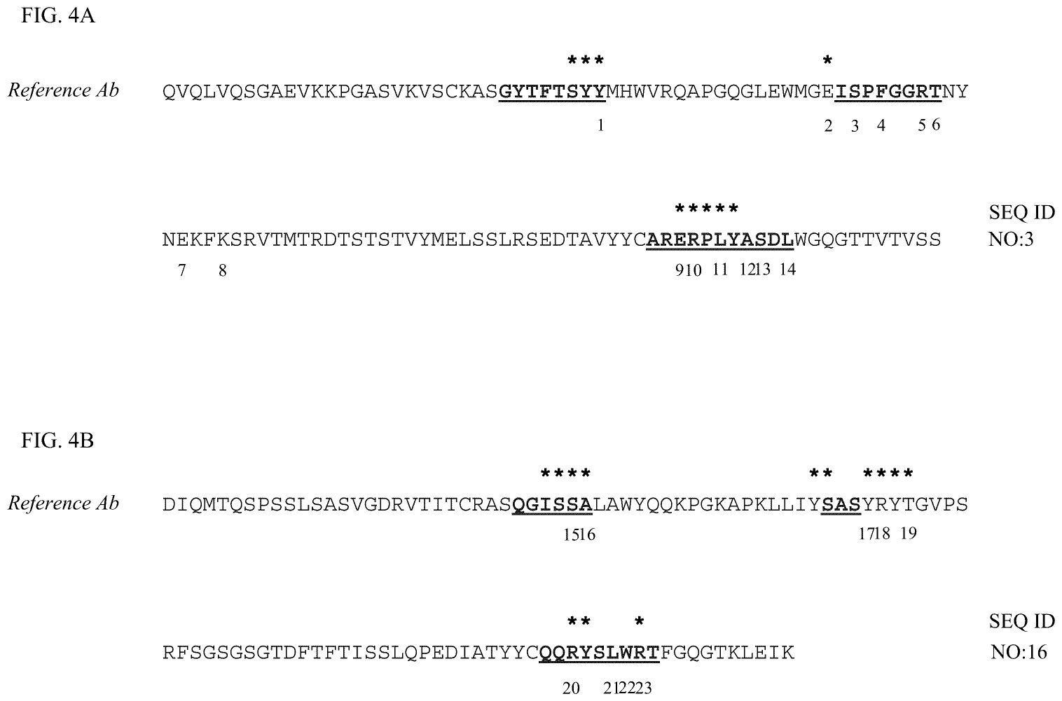

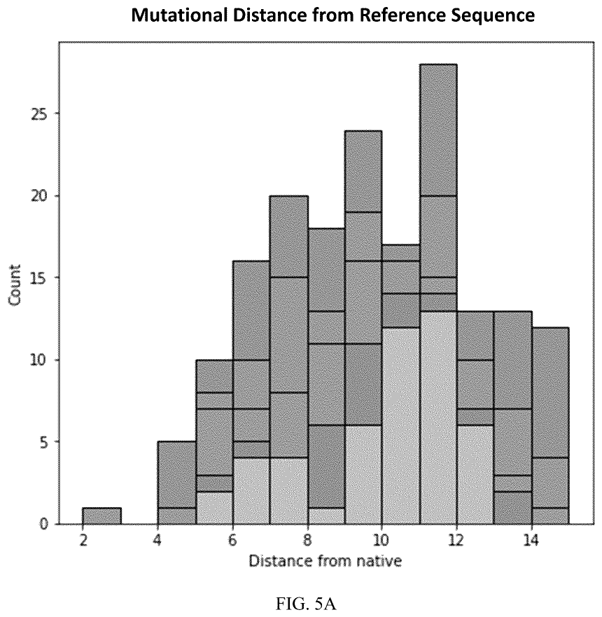

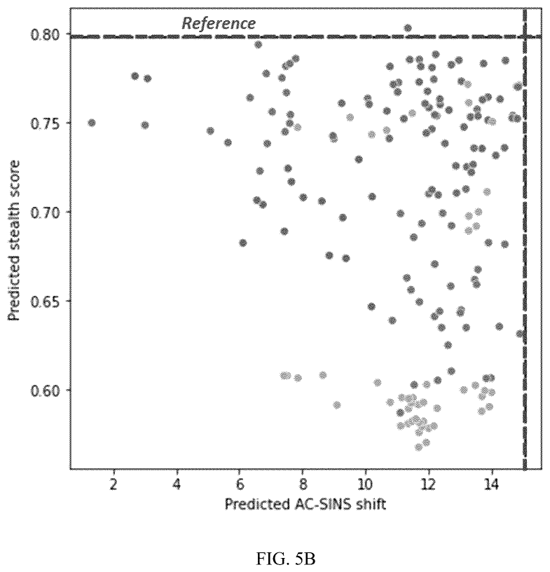

See Table 1 for a listing of SEQ ID NOs: 3-14, Table 2 for a listing of SEQ ID NOs: 16-24, and FIGS. 2A-2C, 3A-3C and 4A-4B for sequences having the paratope residues identified.

The term “paratope” refers to a set of amino acid residues in an antibody or an antigen-binding fragment thereof that contribute to a binding interaction with an epitope of a target protein. The binding interaction can be a hydrogen bond, a salt bridge, a van der Waal interaction, an ionic bond or a combination thereof. A binding interaction may be direct, or indirect, e.g., via a coordinated intermediate molecule, such as an ion or water. The residues of a paratope, in some embodiments, comprise only residues that are part of a defined CDR. In other embodiments, the residues of a paratope further comprise one or more residues that are not part of a defined CDR.

In some embodiments, the paratope comprises positions 31, 32, 33, 50, 99, 100, 101, 102 and 103 of the V_{H} (any one of SEQ ID NO:3-14), and positions 29, 30, 31, 32, 49, 50, 53, 54, 55, 56, 91, 92 and 96 of the V_{L} (any one of SEQ ID NOs: 16-24). In some embodiments, the paratope comprises S31, Y32, Y33, E50, E99, R100, P101, L102 and Y103 of the V_{H} (SEQ ID NO: 3), and I29, S30, S31, A32, Y49, S50, Y53, R54, Y55, T56, R91, Y92 and R96 of the V_{L} (SEQ ID NO:16). See FIGS. 2A-2C, 3A-3C and 4A-4B.

In some embodiments, the paratope of the polypeptide comprises a subset of the CDR residues. In other embodiments, the paratope of the polypeptide further comprises one or more non-CDR paratopic residues.

In some embodiments, the paratope is oriented less than about 5.0 angstroms from an epitope on a target antigen when the polypeptide is bound to the target antigen, e.g., less than about: 4.5, 4.0, 3.5, 3.0, 2.5, 2.4, 2.3, 2.2, 2.1, 2.0, 1.9, 1.8, 1.7, 1.6, 1.5, 1.4, 1.3, 1.2, 1.1, 1.0 or 0.9 angstroms, or about: 0.9-5.0, 0.9-4.8. 1.0-5, 1.0-4.5, 1.0-4.0, 1.0-3.5, 1.1-3.5, 1.1-3.0, 1.2-3.0, 1.2-2.5, 1.3-2.5, 1.3-2.4, 1.4-2.4, 1.4-2.3, 1.5-2.3, 1.5-2.2, 1.6-2.2, 1.6-2.1, 1.7-2.1, 1.7-2.0 or 1.8-2.0 angstroms, from the epitope. In some embodiments, all of the amino acid residues constituting the paratope are oriented less than about 5.0 angstroms from an epitope on a target antigen when the polypeptide is bound to the target antigen. In other embodiments, less than all of the amino acid residues constituting the paratope (e.g., about 40%, 50%, 60%, 70%, 80%, 90% of the amino acid residues) in the paratope are oriented less than about 5.0 angstroms from an epitope on a target antigen when the polypeptide is bound to the target antigen.

In some embodiments, the polypeptide comprising a paratope disclosed herein comprises a V_{H} and a V_{L}. In some embodiments, the paratope residues are contained within the V_{H} and V_{L} of the polypeptide.

In some embodiments, the polypeptide comprises:
a) an immunoglobulin V_{H} comprising a HCDR1, a HCDR2 and a HCDR3 that are at least 50% identical to the HCDR1, HCDR2 and HCDR3, respectively, of at least one sequence set forth in SEQ ID NOs: 3-14;
b) an immunoglobulin V_{L} comprising a LCDR1, a LCDR2 and a LCDR3 that are at least 50% identical to the LCDR1, LCDR2 and LCDR3, respectively, of at least one sequence set forth in SEQ ID NOs: 16-24; or
c) both a) and b).

In some embodiments, the polypeptide comprises a HCDR1 that is at least 50% (e.g., at least 60%, at least 70% or at least 85%) identical to at least one sequence set forth in SEQ ID NOs: 26-28 (i.e., the HCDR1 of at least one sequence set forth in SEQ ID NOs: 3-14). In some embodiments, the polypeptide comprises a HCDR1 comprising at least one amino acid substitution, e.g., 2, 3 or 4 amino acid substitutions, relative to at least one sequence set forth in SEQ ID NO:26-28. In some embodiments, the polypeptide comprises a HCDR1 that is at least 50% (e.g., at least 60%, at least 70% or at least 85%) identical to SEQ ID NO:27 or 28. In some embodiments, the polypeptide comprises a HCDR1 comprising at least one amino acid substitution, e.g., 2, 3 or 4 amino acid substitutions relative to SEQ ID NO:27 or 28. In some embodiments, the polypeptide comprises a HCDR1 that is identical to any one of SEQ ID NOs: 26-28 In some embodiments, the polypeptide comprises a HCDR1 that is identical to SEQ ID NO: 27 or 28. See Table 3 for a listing of SEQ ID NOs: 26-28.

In some embodiments, the polypeptide comprises a HCDR2 that is at least 50% (e.g., at least 60%, at least 70% or at least 85%) identical to at least one sequence set forth in SEQ ID NOs: 30-40 (i.e., the HCDR2 of at least one sequence set forth in SEQ ID NOs: 3-14). In some embodiments, the polypeptide comprises a HCDR2 comprising at least one amino acid substitution, e.g., 2, 3 or 4 amino acid substitutions relative to at least one sequence set forth in SEQ ID NO:30-40. In some embodiments, the polypeptide comprises a HCDR2 that is at least 50% (e.g., at least 60%, at least 70% or at least 85%) identical to at least one sequence set forth in SEQ ID NOs: 31-40. In some embodiments, the polypeptide comprises a HCDR2 comprising at least one amino acid substitution, e.g., 2, 3 or 4 amino acid substitutions, relative to at least one sequence set forth in SEQ ID NOs: 31-40. In some embodiments, the polypeptide comprises a HCDR2 that is identical to any one of SEQ ID NOs: 30-40. In some embodiments, the polypeptide comprises a HCDR2 that is identical to any one of SEQ ID NOs: 31-40. See Table 3 for a listing of SEQ ID NOs: 30-40.

In some embodiments, the polypeptide comprises a HCDR3 that is at least 50% (e.g., at least 55%, at least 60%, at least 65%, at least 70%, at least 80%, at least 85% or at least 90%) identical to at least one sequence set forth in SEQ ID NOs: 42-51 (i.e., the HCDR3 of at least one sequence set forth in SEQ ID NOs: 1-14). In some embodiments, the polypeptide comprises a HCDR3 comprising at least one amino acid substitution, e.g., 2, 3, 4 or 5 amino acid substitutions, relative to at least one sequence set forth in SEQ ID NO:42-51. In some embodiments, the polypeptide comprises a HCDR3 that is at least 50% (e.g., at least 55%, at least 60%, at least 65%, at least 70%, at least 80%, at least 85% or at least 90%) identical to at least one sequence set forth in SEQ ID NOs: 43-51. In some embodiments, the polypeptide comprises a HCDR3 comprising at least one amino acid substitution, e.g., 2, 3, 4 or 5 amino acid substitutions, relative to at least one sequence set forth in SEQ ID NOs: 43-51. In some embodiments, the polypeptide comprises a HCDR3 that is identical to any one of SEQ ID NOs: 42-51. In some embodiments, the polypeptide comprises a HCDR3 that is identical to any one of SEQ ID NOs: 43-51. See Table 3 for a listing of SEQ ID NOs: 42-51.

In some embodiments, the polypeptide comprises a LCDR1 that is at least 50% (e.g., at least 65% or at least 80%) identical to at least one sequence set forth in SEQ ID NO:53-55 (i.e., the LCDR1 of at least one sequence set forth in SEQ ID NOs: 16-24). In some embodiments, the polypeptide comprises a LCDR1 comprising at least one amino acid substitution, e.g., 2, 3 or 4 amino acid substitutions, relative to at least one sequence set forth in SEQ ID NO:53-55. In some embodiments, the polypeptide comprises a LCDR1 that is at least 50% (e.g., at least 65% or at least 80%) identical to the sequence set forth in SEQ ID NO:54 or 55. In some embodiments, the polypeptide comprises a LCDR1 comprising at least one amino acid substitution, e.g., 2, 3 or 4 amino acid substitutions, relative to the sequence set forth in SEQ ID NO: 54 or 55. In some embodiments, the polypeptide comprises a LCDR1 that is identical to any one of SEQ ID NOs: 53-55. In some embodiments, the polypeptide comprises a HCDR1 that is identical to SEQ ID NO:54 or 55. See Table 3 for a listing of SEQ ID NOs: 53-55.

In some embodiments, the polypeptide comprises a LCDR2 that is at least 50% (e.g., at least 65%) identical to SAS. In some embodiments, the polypeptide comprises a LCDR2 comprising an amino acid substitution relative to SAS. In some embodiments, the polypeptide comprises a LCDR2 that is identical to SAS.

In some embodiments, the polypeptide comprises a LCDR3 that is at least 50% (e.g., at least 65%, at least 75% or at least 85%) identical to at least one sequence set forth in SEQ ID NOs: 58-64 (i.e., the LCDR3 of at least one sequence set forth in SEQ ID NOs: 16-24). In some embodiments, the polypeptide comprises a LCDR3 comprising at least one amino acid substitution, e.g., 2, 3 or 4 amino acid substitutions, relative to at least one sequence set forth in SEQ ID NOs: 58-64. In some embodiments, the polypeptide comprises a LCDR3 that is at least 50% (e.g., at least 60%, at least 70%, at least 80% or at least 90%) identical to at least one sequence set forth in SEQ ID NOs: 59-64. In some embodiments, the polypeptide comprises a LCDR3 comprising at least one amino acid substitution, e.g., 2, 3 or 4 amino acid substitutions, relative to at least one sequence set forth in SEQ ID NO:59-64. In some embodiments, the polypeptide comprises a LCDR3 that is identical to any one of SEQ ID NOs: 58-64. In some embodiments, the polypeptide comprises a LCDR3 that is identical to any one of SEQ ID NOs: 59-64. See Table 3 for a listing of SEQ ID NOs: 58-64.

In some embodiments, the polypeptide comprises the HCDR1, HCDR2 and HCDR3, and LCDR1, LCDR2 and LCDR3, of an antibody comprising a V_{H} and a V_{L}, wherein the V_{H} and V_{L} of the antibody are selected from the following V_{H}/V_{L} combinations:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); or
SEQ ID NO:14/SEQ ID NO:16 (AB-11).

In some implementations, the polypeptide further comprises a paratope that is identical to the paratope of an antibody comprising a V_{H} and a V_{L}, wherein the V_{H} and V_{L} of the antibody are selected from the following V_{H}/V_{L} combinations:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); or
SEQ ID NO:14/SEQ ID NO:16 (AB-11).

In some embodiments, the polypeptide comprises:
a) an immunoglobulin V_{H} comprising SEQ ID NO:2, wherein:
   X₁ is not T;
   X₂ is not Y;
   X₃ is not E;
   X₄ is not S;
   X₅ is not P;
   X₆ is not F;
   X₇ is not R;
   X₈ is not T;
   X₉ is not E;
   X₁₀ is not K;
   X₁₁ is not E;
   X₁₂ is not R;
   X₁₃ is not L;
   X₁₄ is not A;
   X₁₅ is not S; or
   X₁₆ is not L, or
   any combination of the foregoing;
b) an immunoglobulin V_{L} comprising SEQ ID NO:15, wherein:
   X₁₇ is not S;
   X₁₈ is not A;
   X₁₉ is not Y;
   X₂₀ is not R;
   X₂₁ is not T;
   X₂₂ is not R;
   X₂₃ is not L;
   X₂₄ is not W; or
   X₂₅ is not R, or
   any combination of the foregoing; or
c) both a) and b).

The sequence identified as SEQ ID NO:2, shown in Table 1, is a consensus V_{H} sequence for SEQ ID Nos: 3-14 herein. The sequence identified as SEQ ID NO:15, shown in Table 3, is a consensus V_{L} sequence for SEQ ID NOs: 16-24 herein.

In some embodiments, the polypeptide comprises an immunoglobulin V_{H} comprising SEQ ID NO:2.

In some embodiments:
X₁ is not T;
X₂ is not Y;
X₃ is not E;
X₄ is not S;
X₅ is not P;
X₆ is not F;
X₇ is not R;
X₈ is not T;
X₉ is not E;
X₁₀ is not K;
X₁₁ is not E;
X₁₂ is not R;
X₁₃ is not L;
X₁₄ is not A;
X₁₅ is not S; or
X₁₆ is not L, or
any combination of the foregoing.

In some embodiments:
X₁ is T or S;
X₂ is Y or W;
X₃ is E or W;
X₄ is S, D, E, F, K, L or R;
X₅ is P or L;
X₆ is F, G or S;
X₇ is R, S or T;
X₈ is T or S;
X₉ is E or Q;
X₁₀ is K or Q;
X₁₁ is E or D;
X₁₂ is R or S;
X₁₃ is L or F;
X₁₄ is A, S or Y;
X₁₅ is S or M; or
X₁₆ is L, I or V, or
any combination of the foregoing.

In some embodiments
X₁ is S;
X₂ is W;
X₃ is W;
X₄ is D, E, F, K, L or R;
X₅ is L;
X₆ is G or S;
X₇ is S or T;
X₈ is S;
X₉ is Q;
X₁₀ is Q;
X₁₁ is D;
X₁₂ is S;
X₁₃ is F;
X₁₄ is S or Y;
X₁₅ is M; or
X₁₆ is I or V, or
any combination of the foregoing.

In some embodiments, X₁ is not T. In some embodiments, X₁ is T or S. In some embodiments, X₁ is S. In some embodiments, X₂ is not Y. In some embodiments, X₂ is Y or W. In some embodiments, X₂ is W. In some embodiments, X₃ is not E. In some embodiments, X₃ is E or W. In some embodiments, X₃ is W. In some embodiments, X₄ is not S. In some embodiments, X₄ is S, D, E, F, K, L or R. In some embodiments, X₄ is D, E, F, K, L or R. In some embodiments, X₅ is not P. In some embodiments, X₅ is P or L. In some embodiments, X₅ is L. In some embodiments, X₆ is not F. In some embodiments, X₆ is F, G or S. In some embodiments, X₆ is G or S. In some embodiments, X₇ is not R. In some embodiments, X₇ is R, S or T. In some embodiments, X₇ is S or T. In some embodiments, X₈ is not T. In some embodiments, X₈ is T or S. In some embodiments, X₈ is S. In some embodiments, X₉ is not E. In some embodiments, X₉ is E or Q. In some embodiments, X₉ is Q. In some embodiments, X₁₀ is not K. In some embodiments, X₁₀ is K or Q. In some embodiments, X₁₀ is Q. In some embodiments, X₁₁ is not E. In some embodiments, X₁₁ is E or D. In some embodiments, X₁₁ is D. In some embodiments, X₁₂ is not R. In some embodiments, X₁₂ is R or S. In some embodiments, X₁₂ is S. In some embodiments, X₁₃ is not L. In some embodiments, X₁₃ is L or F. In some embodiments, X₁₃ is F. In some embodiments, X₁₄ is not A. In some embodiments, X₁₄ is A, S or Y. In some embodiments, X₁₄ is S or Y. In some embodiments, X₁₅ is not S. In some embodiments, X₁₅ is S or M. In some embodiments, X₁₅ is M. In some embodiments, X₁₆ is not L. In some embodiments, X₁₆ is L, I or V. In some embodiments, X₁₆ is I or V.

In some embodiments, the polypeptides comprise the V_{L} comprising SEQ ID NO:15.

In some embodiments:
X₁₇ is not S;
X₁₈ is not A;
X₁₉ is not Y;
X₂₀ is not R;
X₂₁ is not T;
X₂₂ is not R;
X₂₃ is not L;
X₂₄ is not W; or
X₂₅ is not R, or
any combination of the foregoing.

In some embodiments:
X₁₇ is S, G or T;
X₁₈ is A or S;
X₁₉ is Y or N;
X₂₀ is R or L;
X₂₁ is T or S;
X₂₂ is R, H or Y;
X₂₃ is L, N, S, T or V;
X₂₄ is W, A, S or Y; or
X₂₅ is R, L, W or Y, or
any combination of the foregoing.

In some embodiments:
X₁₇ is G or T;
X₁₈ is S;
X₁₉ is N;
X₂₀ is L;
X₂₁ is S;
X₂₂ is H or Y;
X₂₃ is N, S, T or V;
X₂₄ is A, S or Y; or
X₂₅ is L, W or Y, or
any combination of the foregoing.

In some embodiments, X₁₇ is not S. In some embodiments, X₁₇ is S, G or T. In some embodiments, X₁₇ is G or T. In some embodiments, X₁₈ is not A. In some embodiments, X₁₈ is A or S. In some embodiments, X₁₈ is S. In some embodiments, X₁₉ is not Y. In some embodiments, X₁₉ is Y or N. In some embodiments, X₁₉ is N. In some embodiments, X₂₀ is not R. In some embodiments, X₂₀ is R or L. In some embodiments, X₂₀ is L. In some embodiments, X₂₁ is not T. In some embodiments, X₂₁ is T or S. In some embodiments, X₂₁ is S. In some embodiments, X₂₂ is not R. In some embodiments, X₂₂ is R, H or Y. In some embodiments, X₂₂ is H or Y. In some embodiments, X₂₃ is not L. In some embodiments, X₂₃ is L, N, S, T or V. In some embodiments, X₂₃ is N, S, T or V. In some embodiments, X₂₄ is not W. In some embodiments, X₂₄ is W, A, S or Y. In some embodiments, X₂₄ is A, S or Y. In some embodiments, X₂₅ is not R. In some embodiments, X₂₅ is R, L, W or Y. In some embodiments, X₂₅ is L, W or Y.

In some embodiments, the polypeptide comprises a paratope that is at least 80% identical to the paratope of an antibody comprising a V_{H} and a V_{L}, wherein the V_{H} and V_{L} of the antibody are selected from the following V_{H}/V_{L} combinations:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); and
SEQ ID NO:14/SEQ ID NO:16 (AB-11).

In some implementations, the polypeptide comprises a paratope that is at least about: 85%, 90% or 95% identical to the paratope of an antibody comprising any one of the above V_{H}/V_{L} combinations. In some embodiments, the polypeptide comprises a paratope that is identical to the paratope of an antibody comprising any one of the above V_{H}/V_{L} combinations.

In some embodiments, the polypeptide comprises a paratope that differs from the paratope of an antibody comprising any one of the above V_{H}/V_{L} combinations, by substitution of from 1 to 4 (e.g., 1, 2, 3 or 4) paratope residues. In some embodiments, the one or more amino acid substitutions are conservative substitutions. In some embodiments, the one or more amino acid substitutions are highly conservative substitutions.

In some embodiments, the polypeptide comprises:
a) a V_{H} that is at least about 70% identical to at least one sequence set forth in SEQ ID NOs: 3-14;
b) a V_{L} that is at least about 70% identical to at least one sequence set forth in SEQ ID NOs: 16-24; or
c) both a) and b).

In some embodiments, the polypeptide comprises a V_{H} that is at least about 70% identical to at least one sequence (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 sequences) set forth in SEQ ID NOs: 3-14. For example, the V_{H} can be at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to at least one sequence set forth in SEQ ID NOs: 3-14. In some embodiments, the V_{H} is at least about 85%, at least about 90% or at least about 95% identical to at least one sequence set forth in SEQ ID NOs: 3-14.

In some embodiments, the polypeptide comprises a V_{L} that is at least about 70% identical to at least one sequence (e.g., 1, 2, 3, 4, 5, 6, 7, 8, 9, 10, 11 or 12 sequences) set forth in SEQ ID NOs: 16-24. For example, the V_{L} can be at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to at least one sequence set forth in SEQ ID NOs: 16-24. In some embodiments, the V_{L} is at least about 85%, at least about 90% or at least about 95% identical to at least one sequence set forth in SEQ ID NOs: 16-24.

In some embodiments, the polypeptide comprises:
a) a V_{H} that is at least about 1% different from at least one sequence set forth in SEQ ID NOs: 3-14;
b) a V_{L} that is at least about 1% different from at least one sequence set forth in SEQ ID NOs: 16-24; or
c) both a) and b).

In some embodiments, the polypeptide comprises a V_{H} that is at least about 1% different from at least one sequence (e.g., 1, 2, 3, 4, 5, 6, 7, 8, 9, 10 or 11 sequences) set forth in SEQ ID NOs: 3-14. For example, the V_{H} can be at least about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% different from at least one sequence set forth in SEQ ID NOs: 3-14. In some embodiments, the V_{H} is at least about 5%, at least about 10% or at least about 15% different from at least one sequence set forth in SEQ ID NOs: 3-14.

In some embodiments, the polypeptide disclosed herein comprises a V_{L} that is at least about 1% different from at least one sequence (e.g., 1, 2, 3, 4, 5, 6, 7, 8 or 9 sequences) set forth in SEQ ID NOs: 16-24. For example, the V_{L} can be at least about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% different from at least one sequence set forth in SEQ ID NOs: 16-24. In some embodiments, the V_{L} is at least about 5%, at least about 10% or at least about 15% different from at least one sequence set forth in SEQ ID NOs: 16-24.

In some embodiments, the polypeptide comprises:
a) a V_{H} comprising one or more amino acid substitutions relative to at least one sequence set forth in SEQ ID NOs: 3-14;
b) a V_{L} comprising one or more amino acid substitutions relative to at least one sequence set forth in SEQ ID NOs: 16-24; or
c) both a) and b).

In some embodiments, the polypeptide disclosed herein comprises a V_{H} that comprises one or more amino acid substitutions relative to at least one sequence (e.g., 1, 2, 3, 4, 5, 6, 7, 8, 9, 10 or 11 sequences) set forth in SEQ ID NOs: 3-14. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the V_{H} comprises about 1-10 amino acid substitutions, relative to at least one sequence set forth in SEQ ID NOs: 3-14.

In some embodiments, the polypeptide disclosed herein comprises a V_{L} that comprises one or more amino acid substitutions relative to at least one sequence (e.g., 1, 2, 3, 4, 5, 6, 7, 8 or 9 sequences) set forth in SEQ ID NOs: 16-24. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the V_{L} comprises about 1-10 amino acid substitutions, relative to at least one sequence set forth in SEQ ID NOs: 16-24.

In some embodiments, the one or more amino acid substitutions are conservative substitutions. In some embodiments, the one or more amino acid substitutions are highly conservative substitutions.

In some embodiments, the polypeptide comprises:
a) a V_{H} comprising a sequence set forth in any one of SEQ ID NOs: 4-14;
b) a V_{L} comprising a sequence set forth in any one of SEQ ID NOs: 16-24; or
c) both a) and b).

In some embodiments, the polypeptide comprises:
a) a V_{H} comprising a sequence set forth in SEQ ID NO:3;
b) a V_{L} comprising a sequence set forth in any one of SEQ ID NOs: 17-24; or
c) both a) and b).

In some embodiments, the polypeptide disclosed herein comprises a V_{H}/V_{L} combination that is identical to the following V_{H}/V_{L} combination:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); or
SEQ ID NO:14/SEQ ID NO:16 (AB-11).

In some embodiments, the polypeptide disclosed herein comprises:
a) a V_{H} comprising SEQ ID NO:4; and
b) a V_{L} comprising SEQ ID NO:17.

In some embodiments, the polypeptide disclosed herein comprises:
a) a V_{H} comprising SEQ ID NO:5; and
b) a V_{L} comprising SEQ ID NO:18.

In some embodiments, the polypeptide disclosed herein comprises:
a) a V_{H} comprising SEQ ID NO:6; and
b) a V_{L} comprising SEQ ID NO: 16.

In some embodiments, the polypeptide disclosed herein comprises:
a) a V_{H} comprising SEQ ID NO:7 and
b) a V_{L} comprising SEQ ID NO:19.

In some embodiments, the polypeptide disclosed herein comprises:
a) a V_{H} comprising SEQ ID NO:8 and
b) a V_{L} comprising SEQ ID NO: 16.

In some embodiments, the polypeptide disclosed herein comprises:
a) a V_{H} comprising SEQ ID NO:9 and
b) a V_{L} comprising SEQ ID NO:20.

In some embodiments, the polypeptide disclosed herein comprises:
a) a V_{H} comprising SEQ ID NO: 10 and
b) a V_{L} comprising SEQ ID NO:21.

In some embodiments, the polypeptide disclosed herein comprises:
a) a V_{H} comprising SEQ ID NO: 11 and
b) a V_{L} comprising SEQ ID NO:22.

In some embodiments, the polypeptide disclosed herein comprises:
a) a V_{H} comprising SEQ ID NO: 12 and
b) a V_{L} comprising SEQ ID NO:23.

In some embodiments, the polypeptide disclosed herein comprises:
a) a V_{H} comprising SEQ ID NO: 13 and
b) a V_{L} comprising SEQ ID NO:24.

In some embodiments, the polypeptide disclosed herein comprises:
a) a V_{H} comprising SEQ ID NO: 14 and
b) a V_{L} comprising SEQ ID NO:16.

In some embodiments, the polypeptide disclosed herein comprises a V_{H} and V_{L} wherein either or both contain human framework regions.

In some embodiments, the polypeptide disclosed herein is an immunoglobulin molecule, such as an antibody (e.g., a whole antibody, an intact antibody) or an antigen-binding fragment of an antibody.

In some embodiments, the polypeptide disclosed herein is an antibody. As used herein, the term “antibody” refers to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” refers to a full-length antibody comprising two heavy (H) chains and two light (L) chains inter-connected by disulfide bonds or multimers thereof (for example, IgM). Each heavy chain comprises a heavy chain variable region (V_{H}) and a heavy chain constant region (comprising domains CH1, hinge CH2 and CH3). Each light chain comprises a light chain variable region (V_{L}) and a light chain constant region (CL). The V_{H} and the V_{L} regions may be further subdivided into regions of hypervariability, termed complementarity determining regions (CDR), interspersed within framework regions (FR). V_{H} and V_{L} each comprises three CDRs and four FR segments, arranged from the amino-terminus to the carboxy-terminus in the following order: FR1, CDR1, FR2, CDR2, FR3, CDR3, and FR4. The antibody can be of any species, such as a murine antibody, a human antibody or a humanized antibody. Unless otherwise indicated, “antibody” includes an antibody or antigen-binding fragment that has been modified or engineered, or that is a human antibody. Examples of antibodies that have been modified or engineered are chimeric antibodies, humanized antibodies, multiparatopic antibodies, and multispecific antibodies (e.g., bispecific antibodies).

In some embodiments, the polypeptide is a bispecific antibody. In some embodiments, the bispecific antibody comprises a Fc. In other embodiments, the bispecific antibody does not comprise a Fc. Non-limiting example structures of bispecific antibodies include bispecific IgG, IgG appended with an additional antigen-binding moiety, bispecific antibody fragments, bispecific fusion proteins and bispecific antibody conjugates. See, e.g., Spiess et al., Alternative molecular formats and therapeutic applications for bispecific antibodies, Mol Immunol. 67(2 Pt A):95-106 (2015) and Velasquez et al., Redirecting T cells to hematological malignancies with bispecific antibodies, Blood 131(1):30-38 (2018), the contents of which are incorporated herein by reference. In some embodiments, the bispecific antibody is a bispecific T-cell engager. Bispecific T-cell engagers are a class of immunotherapeutic molecules that enhance a patient's immune response to tumor(s) by retargeting T cells to tumor cells. In some embodiments, the bispecific T-cell engager comprises two single-chain variable fragments (scFv) connected in tandem by a flexible linker, a first scFv binds to a T-cell-specific molecule (e.g., CD3), and a second scFv binds to a tumor-associated antigen (e.g., anthrax toxin receptor-like, sodium/hydrogen exchanger 11, disintegrin and metalloproteinase domain-containing protein 2, disintegrin and metalloproteinase domain-containing protein 7, disintegrin and metalloproteinase domain-containing protein 18, Izumo sperm-egg fusion protein 2, sperm acrosome membrane-associated protein 1 or transmembrane protease serine 12).

The extent of the framework region and the CDRs of an antibody can be identified using one of several suitable methodologies that are well known in the art, for example, by the Kabat definition, the Chothia definition, the AbM definition, and/or the contact definition. Publicly and/or commercially available tools for identifying framework and/or CDR regions include, IgBlast (accessible at ncbi.nlm.nih.gov/igblast/), Scaligner (available from drugdesigntech at scaligner.com/), IMGT rules and/or tools (see, for example, imgt.org/IMGTScientificChart/Nomenclature/IMGT-FRCDRdefinition.html, also accessible at imgt.org/), Chothia Canonical Assignment (accessible at bioinf.org.uk/abs/chothia.html), Antigen receptor Numbering And Receptor Calssification (ANARCI, accessible at opig.stats.ox.ac.uk/webapps/newsabdab/sabpred/anarci/), or the Paratome web server (accessible at ofranlab.org/paratome/, see Vered Kunik, et al, Nucleic Acids Research, Volume 40, Issue W1, 1 Jul. 2012, Pages W521-W524).

As used herein, a “CDR” encompasses any CDR defined by an art-recognized method for identifying the CDR residues on an antibody. See, e.g., Kabat, E. A., et al., (1991) Sequences of Proteins of Immunological Interest, Fifth Edition, U.S. Department of Health and Human Services, NIH Publication No. 91-3242, Chothia et al., (1989) Nature 342:877; Chothia, C. et al., (1987) J. Mol. Biol. 196:901-917; Al-lazikani et al., (1997) J. Molec. Biol. 273:927-948; and Almagro, J. Mol. Recognit. 17:132-143 (2004). See also hgmp.mrc.ac.uk and bioinf.org.uk/abs. Two antibodies are determined to have the same CDR as one another with respect to a HCDR1, HCDR2, HCDR3, LCDR1, LCDR2 and/or LCDR3, when the identity of that CDR is determined for both antibodies using the same method.

In some embodiments, the polypeptide disclosed herein is an antigen-binding fragment. The term “antigen-binding fragment” refers to a portion of an immunoglobulin molecule (e.g., an antibody) that retains the antigen binding properties of the parental full-length antibody. Non-limiting examples of antigen-binding fragments include a V_{H} region, a V_{L} region, an Fab fragment, an F(ab′)₂ fragment, an Fd fragment, an Fv fragment, and a domain antibody (dAb) consisting of one V_{H} domain or one V_{L} domain, etc. V_{H} and V_{L} domains may be linked together via a synthetic linker to form various types of single-chain antibody designs in which the V_{H}/V_{L} domains pair intramolecularly, or intermolecularly in those cases when the V_{H} and V_{L} domains are expressed by separate chains, to form a monovalent antigen binding site, such as single chain Fv (scFv) or diabody. In some embodiments, the polypeptide disclosed herein is an antigen binding fragment selected from Fab, F(ab′)₂, Fab′, scFv, or Fv. In some embodiments, the polypeptide is a scFv.

In some embodiments, a polypeptide disclosed herein (e.g., an antibody or antigen-binding fragment) is incorporated into a cell-based therapy. In some embodiments, the polypeptide is an engineered T cell receptor. In some embodiments, the polypeptide is a chimeric antigen receptor (CAR) (e.g., expressed on a T (CAR-T) cell, natural killer (CAR-NK) cell, or macrophage (CAR-M) cell). In some embodiments, the CAR comprises a transmembrane domain and an antigen-recognition moiety, wherein the antigen-recognition moiety binds PCSK9.

In some embodiments, the polypeptide is an antibody mimetic. The term “antibody mimetic” refers to polypeptides capable of mimicking an antibody's ability to bind an antigen, but structurally differ from native antibody structures. Non-limiting examples of antibody mimetics include Adnectins, Affibodies, Affilins, Affimers, Affitins, Alphabodies, Anticalins, Avimers, DARPins, Fynomers, Kunitz domain peptides, monobodies, nanobodies, nanoCLAMPs, and Versabodies.

In some embodiments, the polypeptide further comprises:
a) an antibody heavy chain constant region sequence;
b) an antibody light chain constant region sequence; or
c) both an antibody heavy chain constant region sequence and an antibody light chain constant region sequence.

In some embodiments, the antibody heavy chain constant region is selected from the group consisting of an IgA constant region, an IgD constant region, an IgE constant region, an IgG constant region and an IgM constant region. In some embodiments, the IgG constant region is an IgG1 constant region, an IgG2 constant region, an IgG3 constant region or an IgG4 constant region. In some embodiments, the IgG2 constant region is an IgG2a, an IgG2b constant region or an IgG2c constant region. In some embodiments, the IgA constant region is an IgA1 constant region or an IgA2 constant region. In some embodiments, the antibody heavy chain constant region is an IgG1 constant region (e.g., IGHV5-51). In some embodiments, the antibody heavy chain constant region is an IgG4 constant region.

In some embodiments, the antibody light chain constant region is selected from the group consisting of a κ constant region and a λ constant region. In some embodiments, the antibody light chain constant region is a κ constant region.

In some embodiments, the antibody heavy chain constant region is an IgG1 constant region, and the antibody light chain constant region is a κ constant region. In some embodiments, the antibody heavy chain constant region is an IgG1 constant region, and the antibody light chain constant region is a λ constant region.

In some embodiments, the antibody heavy chain constant region sequence is at least about 60% identical to SEQ ID NO:65. For example, the antibody heavy chain constant region sequence can be at least about: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 65. In some embodiments, the antibody heavy chain constant region sequence is at least about 70% or at least about 80% identical to SEQ ID NO:65. The sequence identified as SEQ ID NO: 65 is shown below:

(SEQ ID NO: 65)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In some embodiments, the antibody heavy chain constant region is an IgG4 constant region, and the antibody light chain constant region is a κ constant region. In some embodiments, the antibody heavy chain constant region is an IgG4 constant region, and the antibody light chain constant region is a λ constant region.

In some embodiments, the antibody heavy chain constant region sequence is at least about 60% identical to SEQ ID NO:66. For example, the antibody heavy chain constant region sequence can be at least about: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 66. In some embodiments, the antibody heavy chain constant region sequence is at least about 70% or at least about 80% identical to SEQ ID NO:66. The sequence identified as SEQ ID NO: 66 is shown below:

(SEQ ID NO: 66)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK.

For additional information on human IgG4 such as clinically tested human IgG4, see, e.g., Silva et al., J Biol Chem. 290(9):5462-9 (2015), the contents of which are incorporated herein in their entirety.

In some embodiments, the antibody light chain constant region sequence is at least about 60% identical to SEQ ID NO:67 or SEQ ID NO:68. For example, the antibody light chain constant region sequence can be at least about: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:67 or SEQ ID NO:68. In some embodiments, the antibody light chain constant region sequence is at least about 70% or at least about 80% identical to SEQ ID NO:67 or SEQ ID NO:68. The sequences identified as SEQ ID NO:67 or SEQ ID NO:68 are shown below:

(SEQ ID NO: 67)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.
(SEQ ID NO: 68)
GQPKANPTVTLFPPSSEELQANKATLVCLISDFYPGAVTVAWKADGSPVK
AGVETTKPSKQSNNKYAASSYLSLTPEQWKSHRSYSCQVTHEGSTVEKTV
APTECS .

In some embodiments, the antibody heavy chain constant region sequence comprises one or more amino acid substitutions relative to SEQ ID NO:65 or SEQ ID NO:66. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the antibody heavy chain constant region sequence comprises about 1-10 amino acid substitutions, relative to SEQ ID NO: 65 or SEQ ID NO:66.

In some embodiments, the antibody light chain constant region sequence comprises one or more amino acid substitutions relative to SEQ ID NO:67 or SEQ ID NO:68. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the antibody light chain constant region sequence comprises about 1-10 amino acid substitutions, relative to SEQ ID NO: 67 or SEQ ID NO:68.

In some embodiments, the one or more amino acid substitutions are conservative substitutions. In some embodiments, the one or more amino acid substitutions are highly conservative substitutions.

In some embodiments, the disclosure provides a polypeptide that specifically binds a PCSK9 (e.g., at one or more human PCSK9 epitope residues identified herein), wherein the polypeptide comprises a paratope that is identical to the paratope of an antibody comprising an immunoglobulin V_{H} and an immunoglobulin V_{L}, wherein the V_{H} and V_{L} of the antibody are selected from the following V_{H}/V_{L} combinations:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); and
SEQ ID NO:14/SEQ ID NO:16 (AB-11).

In some embodiments, the disclosure provides a polypeptide that specifically binds a PCSK9 (e.g., at one or more human PCSK9 epitope residues identified herein), wherein:
a) the polypeptide comprises:
   i. an immunoglobulin V_{H} comprising a HCDR1, a HCDR2 and a HCDR3 that are identical to the HCDR1, HCDR2 and HCDR3, respectively, of any one of SEQ ID NOs: 4-14; and
   ii. an immunoglobulin V_{L} comprising a LCDR1, a LCDR2 and a LCDR3 that are identical to the LCDR1, LCDR2 and LCDR3, respectively, of any one of SEQ ID NOs: 16-24; or
b) the polypeptide comprises:
   i. an immunoglobulin V_{H} comprising a HCDR1, a HCDR2 and a HCDR3 that are identical to the HCDR1, HCDR2 and HCDR3, respectively, of SEQ ID NO:3; and
   ii. an immunoglobulin V_{L} comprising a LCDR1, a LCDR2 and a LCDR3 that are identical to the LCDR1, LCDR2 and LCDR3, respectively, of any one of SEQ ID NOs: 17-24.

In some embodiments, the disclosure provides a polypeptide that specifically binds a PCSK9 (e.g., at one or more human PCSK9 epitope residues identified herein), wherein the polypeptide comprises a V_{H} comprising SEQ ID NO:2, a V_{L} comprising SEQ ID NO:15, or both, wherein:
X₁ is not T;
X₂ is not Y;
X₃ is not E;
X₄ is not S;
X₅ is not P;
X₆ is not F;
X₇ is not R;
X₈ is not T;
X₉ is not E;
X₁₀ is not K;
X₁₁ is not E;
X₁₂ is not R;
X₁₃ is not L;
X₁₄ is not A;
X₁₅ is not S;
X₁₆ is not L;
X₁₇ is not S;
X₁₈ is not A;
X₁₉ is not Y;
X₂₀ is not R;
X₂₁ is not T;
X₂₂ is not R;
X₂₃ is not L;
X₂₄ is not W; or
X₂₅ is not R, or
any combination of the foregoing.

In some embodiments, the polypeptide is an isolated polypeptide. In some embodiments, the isolated polypeptide is recombinantly produced. In some embodiments, the isolated polypeptide is synthetically produced.

In some embodiments, the polypeptide is conjugated to a heterologous moiety. The term “conjugated” refers to attached, via a covalent or noncovalent interaction. Conjugation can employ any of suitable linking agents. Non-limiting examples include peptide linkers, compound linkers, and chemical cross-linking agents.

In some embodiments, the heterologous moiety is a therapeutic agent, a diagnostic agent or a combination thereof. In some embodiments, the heterologous moiety is polyethylene glycol (PEG), hexadecanoic acid, hydrogels, nanoparticles, multimerization domains and carrier peptides.

In some embodiments, the nanoparticle is a lipid nanoparticle. In some embodiments, the nanoparticle is a polymer nanoparticle. In some embodiments, the polymer is an amphiphilic polymer. In other embodiments, the polymer is a hydrophobic or hydrophilic polymer. Non-limiting examples of polymers include poly(lactic acid)-poly(ethylene glycol), poly(lactic-co-glycolic acid)-poly(ethylene glycol), poly(lactic-co-glycolic) acid (PLGA), poly(lactic-co-glycolic acid)-d-α-tocopheryl polyethylene glycol succinate, poly(lactic-co-glycolic acid)-ethylene oxide fumarate, poly(glycolic acid)-poly(ethylene glycol), polycaprolactone-poly (ethylene glycol), or any salts thereof. In some embodiments, the polymer nanoparticle comprises poly(lactic-co-glycolic) acid (PLGA).

In some embodiments, the carrier polypeptide is albumin or an Fc polypeptide.

In some embodiments, the polypeptide:
a) is capable of binding to one or more amino acid residues selected from S1, I2, P3, R42, E43, E45, R85, D86, A87, D215, I217, S220, D222, C223, T225, C226, F227 and S229 of human PCSK9 (SEQ ID NO:1);
b) inhibits PCSK9 binding to LDL-R with an IC₅₀ of 100 nM or less;
c) competes with the Reference Antibody for binding to PCSK9;
d) has a weaker self-association than the Reference Antibody;
e) has a lower serum clearance rate than the Reference Antibody in a subject (e.g., a human);
f) has a longer plasma half-life than the Reference Antibody in a subject (e.g., a human); or
a combination of any of the foregoing.

In some embodiments, the polypeptide is capable of binding to one or more epitope residues of PCSK9, for example, 1, 2, 3, 4, 5, 6, 7, 8, 9, 10, 11, 12, 13, 14, 15, 16, 17 or 18 residues of PCSK9. In some embodiments, the polypeptide is capable of binding to one or more epitope residues (e.g., 1, 2, 3, 4, 5, 6, 7, 8, 9, 10, 11, 12, 13, 14, 15, 16, 17 or all 18 residues) selected from S1, I2, P3, R42, E43, E45, R85, D86, A87, D215, I217, S220, D222, C223, T225, C226, F227 and S229 of SEQ ID NO:1.

In some embodiments, the polypeptide binds PCSK9 or a fragment thereof (e.g., SEQ ID NO: 1) with a binding constant (K_{D}) of about 10 μM or less. As used herein the term “K_{D}),” also referred to as “binding constant,” “equilibrium dissociation constant” or “affinity constant,” is a measure of the extent of a reversible association between two molecular species (e.g., antibody and target protein) and includes both the actual binding affinity as well as the apparent binding affinity. Binding affinity can be determined using methods known in the art including, for example, by measurement of surface plasmon resonance, e.g., using a Biolayer interferometry (Octet, ForteBio) or a surface plasmon resonance (Biacore) system and assay. A reference that compares various surface technologies for measuring binding affinity and kinetics is Yang, D., Singh, A., Wu, H., & Kroe-Barrett, R., Comparison of biosensor platforms in the evaluation of high affinity antibody-antigen binding kinetics, Analytical Biochemistry 508:78-96 (2016), the contents of which are incorporated herein by reference in their entirety.

In some embodiments, the polypeptide binds a PCSK9 or a fragment thereof (e.g., SEQ ID NO:1) with a K_{D} of no more than about: 5 μM, 2 μM, 1 μM, 500 nM, 400 nM, 300 nM, 200 nM, 150 nM, 100 nM, 90 nM, 80 nM, 70 nM, 60 nM, 50 nM, 40 nM, 30 nM, 20 nM, 10 nM, 5 nM, 2 nM, 1 nM, 0.5 nM, 0.2 nM or 0.1 nM. In some embodiments, the polypeptide binds a PCSK9 or a fragment thereof (e.g., SEQ ID NO: 1) with a K_{D} of 50 nM or less. In some embodiments, the polypeptide binds PCSK9 (e.g., SEQ ID NO:1) with a K_{D} of 20 nM or less.

In some embodiments, the polypeptide binds a PCSK9 or a fragment thereof (e.g., SEQ ID NO:1) with a K_{D} of about: 1-200 nM, 1-40 nM, 1-35 nM, 1-30 nM, 1-25 nM, 1-20 nM, 1-15 nM, 2-200 nM, 2-180 nM, 2-40 nM, 2-35 nM, 2-30 nM, 2-25 nM, 2-20 nM, 2-15 nM, 5-180 nM, 5-150 nM, 5-40 nM, 5-35 nM, 5-30 nM, 5-25 nM, 5-20 nM, 5-15 nM, 10-150 nM, 10-120 nM, 10-40 nM, 10-35 nM, 10-30 nM, 10-25 nM, 10-20 nM, 10-15 nM, 15-120 nM, 15-100 nM, 15-40 nM, 15-35 nM, 15-30 nM, 15-25 nM, 15-20 nM, 20-100 nM, 20-90 nM, 20-40 nM, 20-35 nM, 20-30 nM, 20-25 nM, 30-90 nM, 30-80 nM, 30-40 nM, 40-80 nM, 40-70 nM, 10⁻¹⁰-10⁻⁵ M, 10⁻¹⁰-5×10⁻⁶ M, 2×10⁻¹⁰-5×10⁻⁶ M, 2×10⁻¹⁰-2×10⁻⁶ M, 5×10⁻¹⁰-2×10⁻⁶ M, 5×10⁻¹⁰-10⁻⁷ M, 10⁻⁹-10⁻⁷ M, 10⁻⁹-5×10⁻⁸ M, 2×10⁻⁹-5×10⁻⁸ M, 2×10⁻⁹-2×10⁻⁸ M, 5×10⁻⁹-2×10⁻⁸ M or 5×10⁻⁹-10⁻⁸ M.

In some embodiments, the polypeptide competes with the Reference Antibody for binding to a PCSK9 (e.g., wildtype human PCSK9). Techniques and assays for assessing competition between antibodies are known in the art.

In some embodiments, the polypeptide inhibits PCSK9 (e.g., wildtype human PCSK9) binding to LDL-R with an IC₅₀ of about 100 nM or less, e.g., about: 90 nM, 80 nM, 70 nM, 60 nM, 50 nM, 40 nM, 30 nM, 20 nM, 10 nM, 5 nM, 2 nM, 1 nM, 0.5 nM, 0.2 nM or 0.1 nM or less.

In some embodiments, the polypeptide inhibits PCSK9 (e.g., wildtype human PCSK9) binding to LDL-R with an IC₅₀ of: 1-200 nM, 1-40 nM, 1-35 nM, 1-30 nM, 1-25 nM, 1-20 nM, 1-15 nM, 2-200 nM, 2-180 nM, 2-40 nM, 2-35 nM, 2-30 nM, 2-25 nM, 2-20 nM, 2-15 nM, 5-180 nM, 5-150 nM, 5-40 nM, 5-35 nM, 5-30 nM, 5-25 nM, 5-20 nM, 5-15 nM, 10-150 nM, 10-120 nM, 10-40 nM, 10-35 nM, 10-30 nM, 10-25 nM, 10-20 nM, 10-15 nM, 15-120 nM, 15-100 nM, 15-40 nM, 15-35 nM, 15-30 nM, 15-25 nM, 15-20 nM, 20-100 nM, 20-90 nM, 20-40 nM, 20-35 nM, 20-30 nM or 20-25 nM.

In some embodiments, the polypeptide reduces binding of the Reference Antibody to a PCSK9 (e.g., wildtype human PCSK9) by at least about 10%, for example, by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%. In some embodiments, the polypeptide reduces binding of the Reference Antibody to a PCSK9 (e.g., wildtype human PCSK9) by at least about 30%.

In some embodiments, the level of binding between the Reference Antibody and PCSK9 (e.g., wildtype human PCSK9) in the presence of the polypeptide is less than about 90% relative to the level of binding in the absence of the polypeptide, for example, less than about: 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2% or 1%.

In some embodiments, the level of binding between the Reference Antibody and PCSK9 (e.g., wildtype human PCSK9) in the presence of the polypeptide is about 1-90% relative to the level of binding in the absence of the polypeptide, for example, about: 2-90%, 2-85%, 3-85%, 3-80%, 4-80%, 4-75%, 5-75%, 5-70%, 6-70%, 6-65%, 7-65%, 7-60%, 8-60%, 8-55%, 9-55%, 9-50%, 10-50%, 10-45%, 15-45%, 15-40%, 20-40%, 20-35%, 25-35% or 25-30%, relative to the level of binding in the absence of the polypeptide.

In some embodiments, the polypeptide has a weaker self-association than the Reference Antibody, for example, as determined by an affinity-capture self-interaction nanoparticle spectroscopy (AC-SINS) value. The AC-SINS value is the change in maximum absorbance wavelength in the coated-nanoparticle absorption spectra compared to the spectra of the nanoparticle alone. Thus, the greater the change in maximum absorbance wavelength, the more self-interaction of the antibody coated on the nanoparticle. Self-association is an unwanted property that correlates with poor viscosity and poor PK properties. Techniques and assays for assessing self-association of proteins are known in the art. See, e.g., Patro & Przybycien, Biotechnol Bioeng. 52(2):193-203 (1996), the contents of which are incorporated herein in their entirety. In some embodiments, the polypeptide has a weaker self-association than the Reference Antibody.

In some embodiments, the polypeptide has an AC-SINS value of no more than about: 1, 2, 3, 4, 5, 6, 7, 8, 9, 10, 11, 12, 13, 14 or 15. In some embodiments, the polypeptide has an AC-SINS value of no more than about 10. In some embodiments, the polypeptide has an AC-SINS value of about: 1, 2, 3, 4, 5, 6, 7, 8, 9, 10, 11, 12, 13, 14 or 15. In some embodiments, the polypeptide has an AC-SINS value of about 0-15, e.g., 0-12, 0-10, 0-9, 0-8, 0-7, 0-6, 0-5, 0-4, 0-3, 0-2, 0-1, 1-12, 1-10, 1-9, 1-8, 0-7, 1-6, 1-5, 1-4, 1-3, 1-2, 2-12, 2-10, 2-9, 2-8, 2-7, 2-6, 2-5, 2-4, 2-3, 3-12, 3-10, 3-9, 3-8, 3-7, 3-6, 3-5, 3-4, 4-12, 4-10, 4-9, 4-8, 4-7, 4-6, 4-5, 5-12, 5-10, 5-9, 5-8, 5-7 or 5-6. In some embodiments, the polypeptide has an AC-SINS value of about: 0, 1, 2 or 3.

In some embodiments, the polypeptide has an improved developability (e.g. reduced AC-SINS) relative to the Reference Antibody. In some embodiments, the self-association of the polypeptide is at least about 10% lower than that of the Reference Antibody, for example, by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99% lower than that of the Reference Antibody. In some embodiments, the self-association of the polypeptide is at least about 30% lower than that of the Reference Antibody.

In some embodiments, the self-association of the polypeptide is less than about 90% of that of the Reference Antibody, for example, less than about: 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2% or 1% of that of the Reference Antibody.

In some embodiments, the self-association of the polypeptide is about 1-90% relative to that of the Reference Antibody, for example, about: 2-90%, 2-85%, 3-85%, 3-80%, 4-80%, 4-75%, 5-75%, 5-70%, 6-70%, 6-65%, 7-65%, 7-60%, 8-60%, 8-55%, 9-55%, 9-50%, 10-50%, 10-45%, 15-45%, 15-40%, 20-40%, 20-35%, 25-35% or 25-30%, relative to that of the Reference Antibody.

In some embodiments, the reduction in self-association relative to the Reference Antibody is at least about 10%, for example, by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%.

In some embodiments, in a mammalian subject (e.g., a human), the polypeptide induces a serum antibody titer that is at least 2-fold lower than the serum antibody titer induced by the Reference Antibody, for example, at least about: 3-fold lower, 4-fold lower, 5-fold lower, 6-fold lower, 7-fold lower, 8-fold lower, 9-fold lower, 10-fold lower, 20-fold lower, 30-fold lower, 40-fold lower, 50-fold lower, 60-fold lower, 70-fold lower, 80-fold lower, 90-fold lower, 100-fold lower, 200-fold lower, 300-fold lower, 400-fold lower, 500-fold lower, 600-fold lower, 700-fold lower, 800-fold lower, 900-fold lower or 1,000-fold lower than the Reference Antibody.

In some embodiments, the serum antibody titer is detected using an enzyme-linked immunosorbent assay (ELISA). In some embodiments, the mammalian subject has received no prior anti-PCSK9 therapeutic treatments. In some embodiments, the mammalian subject has received at least 1 prior anti-PCSK9 therapeutic treatment (for example, at least 2, 3, 4, 5, or 6 prior anti-PCSK9 therapeutic treatments).

In some embodiments, administration of the polypeptide to a mammalian subject (e.g., a human) induces no immune response in the mammalian subject. In some embodiments, administration of the polypeptide to a mammalian subject induces no detectable immune response in the mammalian subject. In some embodiments, administration of the polypeptide to a mammalian subject induces no antibody-related clinical signs in the mammalian subject. In some embodiments, administration of the polypeptide to a mammalian subject causes no adverse event in the mammalian subject.

In some embodiments, the polypeptide has a lower serum clearance rate than the Reference Antibody in a mammalian subject (e.g., a human).

In some embodiments, the polypeptide has a serum clearance rate that is at least about 10% lower than that of the Reference Antibody, for example, at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99% lower than that of the Reference Antibody. In some embodiments, the polypeptide has a serum clearance rate that is at least about 30% lower than that of the Reference Antibody.

In some embodiments, the polypeptide has a serum clearance rate that is at least about one-fold lower than that of the Reference Antibody, for example, by at least about: 1.5-fold, 2-fold, 2.5-fold, 3-fold, 3.5-fold, 4-fold, 4.5-fold or 5-fold lower than that of the Reference Antibody.

In some embodiments, the polypeptide has a serum clearance rate that is about 20-90% of that of the Reference Antibody, for example, about: 25-90%, 25-85%, 30-85%, 30-80%, 35-80%, 35-75%, 40-75%, 40-70%, 45-70%, 45-65%, 50-65%, 50-60% or 55-60% of that of the Reference Antibody.

In some embodiments, the polypeptide has a plasma half-life that is at least 2 times longer compared to the plasma half-life of the Reference Antibody in a mammalian subject (e.g., a human), for example, at least about: 3 times longer, 4 times longer, 5 times longer, 6 times longer, 7 times longer, 8 times longer, 9 times longer, 10 times longer, 20 times longer, 30 times longer, 40 times longer, 50 times longer, 60 times longer, 70 times longer, 80 times longer, 90 times longer, 100 times longer, 200 times longer, 300 times longer, 400 times longer, 500 times longer, 600 times longer, 700 times longer, 800 times longer, 900 times longer or 1,000 times longer.

In some embodiments, the polypeptide has a plasma half-life that is at least 10% longer compared to that of the Reference Antibody in a mammalian subject (e.g., a human), for example, by at least about: 15%, 20%, 25%, 30%, 35%, 40%, 45%, 50%, 55%, 60%, 65%, 70%, 75%, 80%, 85%, 90%, 95% or 100% longer than that of the Reference Antibody. In some embodiments, the polypeptide of the disclosure has a plasma half-life that is at least 30% longer compared to that of the Reference Antibody in a mammalian subject (e.g., a human).

In some embodiments, the polypeptide has a higher serum concentration than the Reference Antibody in a mammalian subject (e.g., a human).

In some embodiments, the polypeptide has a serum concentration that is at least about one-fold higher than that of the Reference Antibody, for example, by at least about: 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold or 10-fold higher than that of the Reference Antibody.

In some embodiments, the serum concentration of the polypeptide is about 110-1,000% relative to that of the Reference Antibody, for example, about: 115-1,000%, 115-950%, 120-950%, 120-900%, 125-900%, 125-850%, 130-850%, 130-800%, 135-800%, 135-750%, 140-750%, 140-700%, 145-700%, 145-650%, 150-650%, 150-600%, 155-600%, 155-550%, 160-550%, 160-500%, 165-500%, 165-450%, 170-450%, 170-400%, 175-400%, 175-350%, 180-350%, 180-300%, 185-300%, 185-250%, 190-250% or 190-200%, relative to that of the Reference Antibody.

The foregoing properties of certain embodiments of the polypeptides provided herein can produce clinically meaningful benefits as further discussed, infra, e.g., in paragraphs [00197]-[00244].

### Fusion Proteins

In some embodiments, the disclosure provides a fusion protein comprising one or more of the polypeptides described herein.

The term “fusion protein” refers to a synthetic, semi-synthetic or recombinant single protein molecule. A fusion protein can comprise all or a portion of two or more different proteins and/or polypeptides that are attached by covalent bonds (e.g., peptide bonds).

Fusion proteins of the disclosure can be produced recombinantly or synthetically, using routine methods and reagents that are well known in the art. For example, a fusion protein of the disclosure can be produced recombinantly in a suitable host cell (e.g., bacteria) according to methods known in the art. See, e.g., Current Protocols in Molecular Biology, Second Edition, Ausubel et al. eds., John Wiley & Sons, 1992; and Molecular Cloning: a Laboratory Manual, 2nd edition, Sambrook et al., 1989, Cold Spring Harbor Laboratory Press. For example, a nucleic acid molecule comprising a nucleotide sequence encoding a fusion protein described herein can be introduced and expressed in suitable host cell (e.g., E. coli), and the expressed fusion protein can be isolated/purified from the host cell (e.g., in inclusion bodies) using routine methods and readily available reagents. For example, DNA fragments coding for different protein sequences (e.g., a light-responsive domain, a heterologous peptide component) can be ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992).

### Nucleic Acids, Expression Vectors, Expression Host Cells

In some embodiments, the disclosure provides one or more polynucleotides encoding any one of the polypeptides or fusion proteins described herein. In some embodiments, the polypeptide or fusion protein of the disclosure is encoded by a single polynucleotide. In some embodiments, the polypeptide or fusion protein of the disclosure is encoded by multiple polynucleotides. Non-limiting examples of polynucleotides include linear deoxyribonucleic acid (DNA), linear ribonucleic acid (RNA), circular DNA and circular RNA, etc.

In some embodiments, the polynucleotide comprises a nucleotide sequence that is codon-optimized for a chosen host cell.

In some embodiments, the disclosure provides an expression vector comprising any one or more of the polynucleotides described herein.

The term “expression vector” refers to a replicable nucleic acid from which one or more proteins can be expressed when the expression vector is transformed into a suitable expression host cell.

In some embodiments, the expression vector further comprises an expression control polynucleotide sequence operably linked to the polynucleotide, a polynucleotide sequence encoding a selectable marker, or both. In some embodiments, the expression control polynucleotide sequence comprises a promoter sequence, an enhancer sequence, or both. In some embodiments, the expression control polynucleotide sequence comprises an inducible promoter sequence. The term “promoter” refers to a region of DNA to which RNA polymerase binds and initiates the transcription of a gene. The term “operably linked” means that the nucleic acid is positioned in the recombinant polynucleotide, e.g., vector, in such a way that enables expression of the nucleic acid under control of the element (e.g., promoter) to which it is linked. The term “selectable marker element” is an element that confers a trait suitable for artificial selection. Selectable marker elements can be negative or positive selection markers.

In some embodiments, the disclosure provides an expression host cell comprising any one or more of the polynucleotides or expression vectors described herein.

The term “expression host cell” refers to a cell useful for receiving, maintaining, reproducing and amplifying a vector.

Non-limiting examples of expression host cells include mammalian cells such as hybridoma cells, Chinese hamster ovary (CHO) cells, COS cells, human embryonic kidney (HEK), yeast cells such as Pichia pastoris cells, or bacterial cells such as DH5α, etc.

### Compositions

In some embodiments, the disclosure provides a composition comprising any one of the polypeptides or fusion proteins described herein. In some embodiments, the composition is a pharmaceutical composition.

In some embodiments, the composition (e.g., pharmaceutical composition) further comprises pharmaceutically acceptable carriers, excipients, stabilizers, diluents or tonifiers (Remington's Pharmaceutical Sciences 16th edition, Osol, A. Ed. (1980)). Suitable pharmaceutically acceptable carriers, excipients, or stabilizers are nontoxic to recipients at the dosages and concentrations employed. Non-limiting examples of pharmaceutically acceptable carriers, excipients, stabilizers, diluents or tonifiers include buffers (e.g., phosphate, citrate, histidine), antioxidants (e.g., ascorbic acid or methionine), preservatives, proteins (e.g., serum albumin, gelatin or immunoglobulins); hydrophilic polymers, amino acids, carbohydrates (e.g., monosaccharides, disaccharides, glucose, mannose or dextrins); chelating agents (e.g., EDTA), sugars (e.g., sucrose, mannitol, trehalose or sorbitol), salt-forming counter-ions (e.g., sodium), metal complexes (e.g., Zn-protein complexes); non-ionic surfactants (e.g., Tween), PLURONICS™ and polyethylene glycol (PEG).

In some embodiments, the composition (e.g., pharmaceutical composition) of the disclosure is formulated for a suitable administration schedule and route. Non-limiting examples of administration routes include oral, rectal, mucosal, intravenous, intramuscular, subcutaneous and topical, etc. In some embodiments, the composition (e.g., pharmaceutical composition) of the disclosure is stored in the form of an aqueous solution or a dried formulation (e.g., lyophilized).

In some embodiments, the composition is formulated to be administered by infusion (e.g., intravenous infusion).

In some embodiments, the composition is formulated to be administered with a second therapeutic agent as a combination therapy.

In some embodiments, the composition comprises a polypeptide comprising a V_{H}/V_{L} combination of:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); or
SEQ ID NO:14/SEQ ID NO:16 (AB-11), or
a variant thereof.

In some embodiments, the composition comprises a polypeptide comprising a V_{H}/V_{L} combination of:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); or
SEQ ID NO:14/SEQ ID NO:16 (AB-11).

### Methods of Use

In some embodiments, the disclosure provides a method of modulating (e.g., decreasing, increasing) PCSK9 activity in a mammalian cell, comprising contacting the cell with any one of the polypeptides, fusion proteins or compositions described herein. In some embodiments, the method of modulating is a method of decreasing, or inhibiting, PCSK9 activity (e.g., binding to LDL-R) in a mammalian cell.

In some embodiments, the disclosure provides a method of treating a subject in need thereof, comprising administering to the subject an effective amount of any one of the compositions described herein (e.g., pharmaceutical compositions comprising a pharmaceutically acceptable carrier and, wherein as an active ingredient, any one of the polypeptides or fusion proteins described herein).

In some embodiments, the disclosure provides a method of treating a disease or condition associated with an increased LDL level in the blood in a subject in need thereof, comprising administering to the subject an effective amount of any one of the compositions (e.g., pharmaceutical compositions comprising a pharmaceutically acceptable carrier and, wherein as an active ingredient, any one of the polypeptides or fusion proteins described herein) described herein.

The term “subject” or “patient” refers to an animal (e.g., a mammal). In some embodiments, the subject is a mammal. In some embodiments, the subject is a mammal selected from the group consisting of a dog, a cat, a mouse, a rat, a hamster, a guinea pig, a horse, a pig, a sheep, a cow, a chimpanzee, a macaque, a cynomolgus, and a human. In some embodiments, the subject is a primate. In some embodiments, the subject is a human.

In some embodiments, the subject is a pediatric patient. In some embodiments, the subject is a juvenile patient. In some embodiments, the subject is an adult patient.

In some embodiments, the subject is two years of age or older, for example, at least: 3, 4, 5, 6, 7, 8, 9, 10, 11, 12 years of age or older. In some embodiments, the subject is 4 years of age or older. In some embodiments, the subject is 5 years of age or older. In some embodiments, the subject is 6 years of age or older. In some embodiments, the subject is 12 years of age or older. In some embodiments, the subject is 18 years of age or older. In some embodiments, the subject is 18-75 years of age. In some embodiments, the subject is 40 years of age or older, e.g., at least: 45, 50, 55, 60, 65, 70, 75, 80, 85 or 90 years old.

A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

In some embodiments, the subject has a disease or condition associated with an increased LDL level in the blood. In some embodiments, the subject has been diagnosed with a disease or condition associated with an increased LDL level in the blood. In other embodiments, the subject is at risk of developing a disease or condition associated with an increased LDL level in the blood.

In some embodiments, the disease or condition associated with an increased LDL level in the blood is atherosclerosis, a cardiovascular disease, a coronary heart disease (CVD), dyslipidemia, hypercholesterolemia, hyperlipidemia, or a combination thereof. In some embodiments the disease or conditions is associated with heterozygous familial hypercholesterolemia (HeFH) or homozygous familial hypercholesterolemia (HoFH), or a combination thereof.

In some embodiments, the disease or condition is associated with an increased LDL-C levels. In some embodiments, the subject is at risk of myocardial infarction, stroke, unstable angina, coronary revascularization, or combinations thereof. In some embodiments the disease or condition is established cardiovascular disease (CVD), ischemic heart diseases, or coronary artery disease, or combinations thereof. In some embodiments, the subject may be undergoing a concurrent therapy. Non-limiting examples of a concurrent therapy include one or more of: modified diet, a statin, or cholesterol absorption inhibitor.

In some embodiments, the dyslipidemia is mixed dyslipidemia.

In some embodiments, the hypercholesterolemia is heterozygous familial hypercholesterolemia (HetFH). In some embodiments, the hypercholesterolemia is homozygous familial hypercholesterolemia (HoFH).

In some embodiments, the hyperlipidemia is heterozygous familial hyperlipidemia. In some embodiments, the hyperlipidemia is homozygous familial hyperlipidemia. In some embodiments, the hyperlipidemia is non-familial hyperlipidemia. In some embodiments, the hyperlipidemia is primary hyperlipidemia.

“A therapeutically effective amount,” “an effective amount” or “an effective dosage” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

In some embodiments, the amount (e.g., therapeutically effective amount) of the polypeptide is sufficient to reduce binding between PCSK9 and LDL-R.

In some embodiments, the amount (e.g., therapeutically effective amount) of the polypeptide is sufficient to reduce binding between PCSK9 and LDL-R by at least about 10%, for example, by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%. In some embodiments, the amount (e.g., therapeutically effective amount) of the polypeptide is sufficient to reduce binding between PCSK9 and LDL-R by at least about 30%.

In some embodiments, the level of binding between PCSK9 and LDL-R in the presence of the polypeptide is less than about 90% relative to the level of binding in the absence of the polypeptide, for example, less than about: 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2% or 1%.

In some embodiments, the level of binding between PCSK9 and LDL-R in the presence of the polypeptide is about 1-90% relative to the level of binding in the absence of the polypeptide, for example, about: 1-20%, 1-15%, 1-10%, 1-5%, 1-4%, 1-3%, 1-2%, 2-90%, 2-85%, 2-20%, 2-15%, 2-10%, 2-5%, 2-4%, 2-3%, 3-85%, 3-80%, 3-20%, 3-15%, 3-10%, 4-80%, 4-20%, 4-15%, 4-10%, 4-75%, 5-75%, 5-70%, 5-20%, 5-15%, 5-10%, 6-70%, 6-65%, 6-20%, 6-15%, 6-10%, 7-65%, 7-60%, 8-60%, 7-20%, 7-15%, 7-10%, 8-55%, 8-20%, 8-15%, 9-55%, 9-50%, 9-20%, 9-15%, 10-50%, 10-45%, 10-20%, 10-15%, 15-45%, 15-40%, 20-40%, 20-35%, 25-35% or 25-30%, relative to the level of binding in the absence of the polypeptide.

In some embodiments, the amount (e.g., therapeutically effective amount) of the polypeptide is sufficient to reduce binding between PCSK9 and LDL-R.

In some embodiments, the amount (e.g., therapeutically effective amount) of the polypeptide is sufficient to reduce LDL level in the blood.

In some embodiments, the amount (e.g., therapeutically effective amount) of the polypeptide is sufficient to reduce LDL level in the blood by at least about 10%, for example, by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%. In some embodiments, the amount (e.g., therapeutically effective amount) of the polypeptide is sufficient to reduce level in the blood by at least about 30%.

In some embodiments, the LDL level in the blood in the presence of the polypeptide is less than about 90% relative to the level of binding in the absence of the polypeptide, for example, less than about: 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2% or 1%.

In some embodiments, the LDL level in the blood in the presence of the polypeptide is about 1-90% relative to the level of binding in the absence of the polypeptide, for example, about: 1-20%, 1-15%, 1-10%, 1-5%, 1-4%, 1-3%, 1-2%, 2-90%, 2-85%, 2-20%, 2-15%, 2-10%, 2-5%, 2-4%, 2-3%, 3-85%, 3-80%, 3-20%, 3-15%, 3-10%, 4-80%, 4-20%, 4-15%, 4-10%, 4-75%, 5-75%, 5-70%, 5-20%, 5-15%, 5-10%, 6-70%, 6-65%, 6-20%, 6-15%, 6-10%, 7-65%, 7-60%, 8-60%, 7-20%, 7-15%, 7-10%, 8-55%, 8-20%, 8-15%, 9-55%, 9-50%, 9-20%, 9-15%, 10-50%, 10-45%, 10-20%, 10-15%, 15-45%, 15-40%, 20-40%, 20-35%, 25-35% or 25-30%, relative to the level in the absence of the polypeptide.

An effective amount of an agent to be administered can be determined by a clinician of ordinary skill using the guidance provided herein and other methods known in the art. Relevant factors include the given agent, the pharmaceutical formulation, the route of administration, the type of disease or disorder, the identity of the subject (e.g., age, sex, weight) or host being treated, and the like. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

Desired response or desired results include effects at the cellular level, tissue level, or clinical results. As such, “a therapeutically effective amount” or synonym thereto depends upon the context in which it is being applied. For example, in some embodiments it is an amount of the composition sufficient to achieve a treatment response as compared to the response obtained without administration of the composition. In other embodiments, it is an amount that results in a beneficial or desired result in a subject as compared to a control. As defined herein, a therapeutically effective amount of a composition of the present disclosure may be readily determined by one of ordinary skill by routine methods known in the art. Dosage regimen and route of administration may be adjusted to provide the optimum therapeutic response.

In some embodiments, the method is used for prophylactic therapy. In some embodiments, the effective dosage is sufficient to prevent the subject of developing a disease or condition (e.g., a chronic inflammatory disease).

The term “treating” or “treatment” refers to the medical management of a subject with the intent to improve, ameliorate, stabilize (i.e., not worsen), prevent or cure a disease, pathological condition, or disorder-such as the particular indications exemplified herein. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

In some embodiments, the method increases overall survival (e.g., relative to untreated or treated with the Reference Antibody) by at least about 10%, e.g., by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%.

In some embodiments, the method increases overall survival (e.g., relative to untreated or treated with the Reference Antibody) by at least about: 1 month, e.g., by at least about: 2, 3, 4, 5, 6, 7, 8, 9, 10, 11, 12, 18, 24, 30, 36, 42, 48, 54, 60, 66, 72, 78 or 84 months. In some embodiments, the method increases overall survival by about: 1 month, e.g., by about: 2, 3, 4, 5, 6, 7, 8, 9, 10, 11, 12, 18, 24, 30, 36, 42, 48, 54, 60, 66, 72, 78 or 84 months. In some embodiments, the method increases overall survival by about: 1-60 months, e.g., by about: 2-84, 2-78, 3-78, 3-72, 4-72, 4-66, 5-66, 5-60, 6-60, 6-54, 7-54, 7-48, 8-48, 8-42, 9-42, 9-36, 10-36, 10-30, 11-30, 11-24, 12-24 or 12-18 months.

A therapeutic agent described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen.

In some embodiments, a polypeptide, polynucleotide, vector, host cell, composition, or pharmaceutical composition disclosed herein is administered to a subject in combination with one or more additional therapeutic agents (e.g., concurrently or sequentially with one or more additional therapeutic agents). In some embodiments, a subject has been previously treated with one or more therapeutic agents prior to being administered a polypeptide, composition, or pharmaceutical composition disclosed herein. In some embodiments, the method further comprises administering a therapeutically effective amount of one or more additional therapeutic agents to the subject at the same time as, prior to, or following administration of a polypeptide, composition, or pharmaceutical composition disclosed herein.

In some embodiments, the method further comprises administering a therapeutically effective amount of a second therapeutic agent to the subject.

Administration of two or more therapeutic agents encompasses co-administration of the therapeutic agents in a substantially simultaneous manner, such as in a pharmaceutical combination. Alternatively, such administration encompasses co-administration in multiple containers, or separate containers (e.g., capsules, powders, and liquids) for each therapeutic agent. Such administration also encompasses use of each type of therapeutic agent in a sequential manner, either at approximately the same time or at different times. The composition described herein and the additional therapeutic agent(s) can be administered via the same administration route or via different administration routes. In some embodiments, the subject is receiving concurrent or prior therapy selected from a diet, a static, or a cholesterol blocker, or any combination thereof.

In some embodiments, the disclosure provides a method of preventing a disease or condition associated with an increased LDL level in the blood in a subject in need thereof, comprising administering to the subject an effective amount of a pharmaceutical composition comprising a pharmaceutically acceptable carrier and, wherein as an active ingredient, any one of the polypeptides or fusion proteins described herein.

In some embodiments, the disclosure provides a method of inhibiting (e.g., reducing) a disease or condition associated with an increased LDL level in the blood in a subject in need thereof, comprising administering to the subject an effective amount of a pharmaceutical composition comprising a pharmaceutically acceptable carrier and, wherein as an active ingredient, any one of the polypeptides or fusion proteins described herein.

In some embodiments, the disclosure provides a method of modulating (e.g., decreasing) PCSK9 activity in a subject in need thereof, comprising administering to the subject an effective amount of a pharmaceutical composition comprising a pharmaceutically acceptable carrier and, wherein as an active ingredient, any one of the polypeptides or fusion proteins described herein.

In some embodiments, the disclosure provides a method of modulating (e.g., decreasing) PCSK9 activity in a mammalian cell, comprising contacting the mammalian cell with an effective amount of any one of the polypeptides or fusion proteins described herein.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the disclosure are applicable to all of the other aspects of the disclosure mutatis mutandis and, without limitation, are exemplified by the dependent claims and also encompass combinations and permutations of individual features (e.g., elements, including numerical ranges and exemplary embodiments) of particular embodiments and aspects of the disclosure, including the working examples. For example, particular experimental parameters exemplified in the working examples can be adapted for use in the claimed disclosure piecemeal without departing from the disclosure. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the disclosure, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed disclosure by, inter alia, a negative proviso or disclaimer of the feature or combination of features.

### Exemplification

Immunogenicity of biotherapeutic proteins can result in emergence of anti-drug antibodies (ADAs), drug neutralization, hypersensitivity and ultimately failure in the clinic. Immune risk factors (such as foreign peptide presentation on MHC II) and developability liabilities (such as self-association, aggregation, or poly-specificity) can significantly increase the occurrence of immunogenicity. To produce high-quality drug candidates during the earliest stages of design, machine learning (ML) predictors and an ensemble of protein sequence generation methods were utilized to co-optimize for developability, immunogenicity and function. Here these approaches were utilized to demonstrate simultaneous improvements in the developability and immunogenicity of the Reference Antibody, a poorly behaved molecule with high rates of ADAs in the clinic.

### Example 1. PCSK9 Binding Polypeptides

A set of 179 variants of the Reference Antibody were generated, each falling into individual computational design groups. Sequence variations were restricted to CDR regions and non-germline framework residues of the antibody, with up to 14 mutations away from Reference Antibody as shown in FIG. 5A. Non-germline residues were defined as framework mismatches between the Reference Antibody and its closest human germline sequences (IGHV1-46*01, IGHJ6*01, IGKV1-12*01, and IGKJ2*01). All variants were predicted to have improved developability (e.g., reduced AC-SINS) and decreased immunogenicity risk (e.g. reduced number of peptides predicted to be presented on MHC II) as shown in FIG. 5B. Candidates were expressed as full-length IgGs and evaluated for self-association, binding to PCSK9 antigen, ability to disrupt PCSK9 binding to LDL-R and internalization by DCs (as a proxy for immunogenicity risk). All 179 variants were evaluated for their propensity to self-associate via an AC-SINS assay.

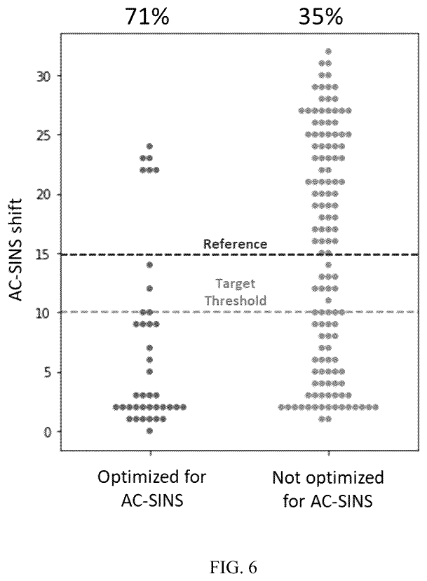

As shown in FIG. 6, for those variants computationally designed to specifically optimize towards improvements in AC-SINS, 71% of variants showed improved (or decreased) AC-SINS compared to the Reference Antibody. In comparison, those variants not specifically optimized towards improvements in AC-SINS only showed 35% of designs with improvements compared to the reference. All variants were evaluated for relative binding to PCSK9 via a DELFIA immunoassay (PerkinElmer, Cat. #1244-330). All positive binders demonstrating improved developability measures, which included 63 variants and the reference control, were assessed for function via a PCSK9 biochemical disruption assay (BPS Biosciences, Cat. #72010).

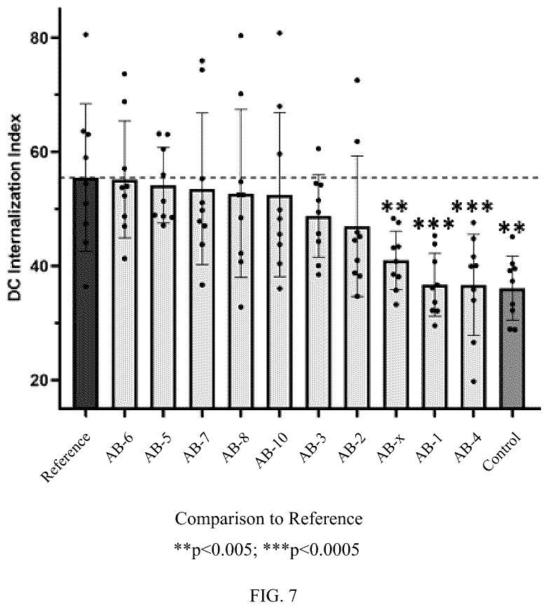

Ten of the 63 variants exhibited functional activity to disrupt the interaction of PCSK9 with its ligand LDL-R, as shown in Table 1. The 10 functional variants were then assessed via an in vitro dendritic cell (DC) immunogenicity assay which measures the relative uptake or internalization of the antibody by DCs (Wen Y. et al AAPS J. 2020), as shown in FIG. 7. All functional variants exhibited similar or reduced DC uptake, with three of the variants showing significant reductions in uptake with levels more similar to the control antibody (Evolocumab) low immunogenicity reference. This demonstration shows the ability of an ML guided approach to produce drug candidates with superior developability and immunogenicity profiles from the initial generation step, potentially dramatically reducing downstream time and cost required to optimize drug candidates.

Table 4 shows the results of antibody variants and controls binding to target antigen PCSK9 and ability to disrupt PCSK9 binding to the LDL-R ligand in functional assays. The antibody identification number and distance from the Reference Antibody are noted. The relative binding of each variant to PCSK9 as measured by DELIFA immunoassay is shown. The functionality, or ability of a variant to disrupt PCSK9 binding to LDL-R is shown with “Yes” indicating a positive signal for disruption and “Not Determined” indicating that the antibody was not evaluated via this assay. For those antibodies demonstrating functional activity, a IC₅₀ value is shown, with “>53” indicating that the antibody demonstrated positive signal for disruption but failed to reach its IC₅₀ value at the concentrations tested.

### Example 2. Peptide Presentation on MHC-II

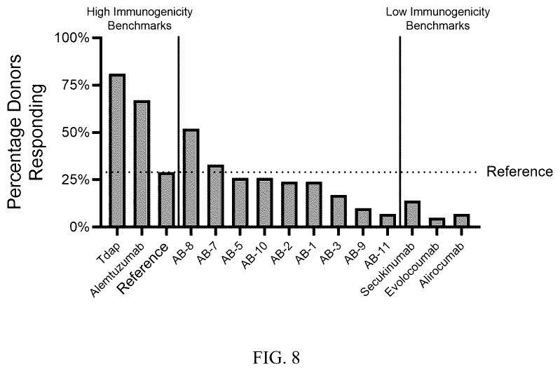

To evaluate MHC-II peptide presentation, the Reference Antibody and two variant antibodies, AB-8 and AB-11, were chosen for assessment via MCH-II associated peptide proteomics (MAPPs) (Barra C. et al., Frontiers in Immunology 2020). AB-11 was chosen as it demonstrated reduced T cell antigenicity as shown in FIG. 8, while AB-8 was chosen as it showed increased antigenicity. In brief, recombinantly expressed antibodies were loaded onto PBMC-derived DCs for 24 hours, with cells frozen down for further processing. Cells were lysed, and MHC-II molecules isolated via immunoprecipitation with a pan anti-HLA II antibody (clone IVA12). Peptides were then eluted off of MHC-II molecules using an acid wash and analyzed by LC/LC mass spec. Detected peptides were mapped back to the protein loaded on to the DCs and the relative number of peptides presented were compared between antibody variants and reference control.

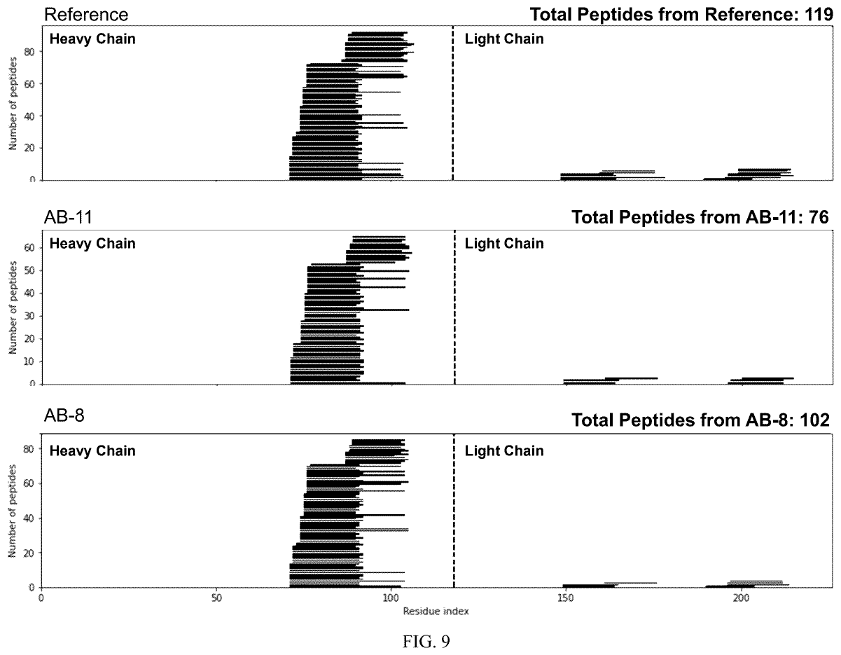

As shown in FIG. 9, fewer total unique peptides were presented from AB-11 than for either AB-8 or the Reference Antibody.

### Example 3. Reduction in Immunogenicity

To indirectly demonstrate reduced peptide presentation, but directly demonstrate reduction in immunogenicity, top variants, the Reference Antibody and controls were assessed via an in vitro T cell proliferation assay (Anja T. B. et al., Frontiers in Immunology 2017). In brief, PBMC-derived DCs were loaded with antibody variants or reference controls for 24 hours and stimulated with Lipopolysaccharide. CD⁴⁺ T cells were isolated from the same donors from which the DCs were derived and labeled with Celltrace Violet proliferation dye (Thermofisher, Cat. C34557). CD4⁺ T cells were co-cultured with the loaded DCs for 6-7 days. The percentages of CD4⁺ T cells that proliferated when exposed to the antibody loaded DCs were assessed via flow cytometry. A donor was considered to be a responder if it exhibited a higher than two-fold increase in the percentage of proliferated CD4⁺ T cells when stimulated by DCs loaded with the test article as compared to unloaded DC controls from the same donor. The percentage of responding donors were then compared across all test articles versus the Reference antibody, high immunogenicity benchmark controls (Alemtuzumab and Tdap vaccine) and low immunogenicity benchmark controls (Secukinumab, Evolocumab and Alirocumab).

As shown in FIG. 8, out of the nine variants evaluated, three demonstrated a reduced (AB-3, AB-9 and AB-11), five exhibited a similar (AB-7, AB-5, AB-10, AB-2 and AB-1), and one showed an increased (AB-8) percentage of donors responding compared to the Reference Antibody. The three variants exhibiting reductions in the percentage of donors responding (AB-3, AB-9 and AB-11), also demonstrated similar levels of immunogenicity to the low immunogenicity benchmark control molecules. Since these molecules exhibit minimal immunogenicity in clinical trials, these results support the conclusion that the relative immunogenicity risk of AB-3, AB-9 and AB-11 has been reduced compared to the Reference Antibody.

The above demonstrations would show the ability of a machine learning (ML) guided approach to produce drug candidates with superior developability and reduced immunogenicity risk profiles from the initial generation step.

### REFERENCES

1. Bergeron N, Phan B A, Ding Y, Fong A, Krauss R M. Circulation. 132(17):1648-66 (2015).
2. Ridker P M, Amarenco P, Brunell R, Glynn R J, Jukema J W, Kastelein J J, Koenig W, Nissen S, Revkin J, Santos R D, Schwartz P F, Yunis C, Tardif J C. Am Heart J. 178:135-44 (2016).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

TABLE 1
VH Amino Acid Sequences

| SEQ | | |
|---|---|---|
| ID | | Amino Acid Sequence |
| NO: 2 | Consensus | QVQLVQSGAEVKKPGASVKVSCKASGYX1FTSYX2MHWVRQAPGQGLEW |
| | | MGX₃IX₄X₅X₆GGX₇X₈NYNX₉KFX₁₀SRVTMTRDTSTSTVYMELSSLRSEDTA |
| | | VYYCARX₁₁X₁₂PX₁₃YX₁₄X₁₅DX₁₆WGQGTTVTVSS |
| NO: 3 | Reference | QVQLVQSGAEVKKPGASVKVSCKASGYTFTSYYMHWVRQAPGQGLEWM |
| | | GEISPFGGRTNYNEKFKSRVTMTRDTSTSTVYMELSSLRSEDTAVYYCARE |
| | | RPLYASDLWGQGTTVTVSS |
| NO: 4 | AB-1 | QVQLVQSGAEVKKPGASVKVSCKASGYTFTSYYMHWVRQAPGQGLEWM |
| | | GEILPGGGTTNYNQKFKSRVTMTRDTSTSTVYMELSSLRSEDTAVYYCARE |
| | | SPLYASDIWGQGTTVTVSS |
| NO: 5 | AB-2 | QVQLVQSGAEVKKPGASVKVSCKASGYTFTSYYMHWVRQAPGQGLEWM |
| | | GWIEPFGGRTNYNEKFKSRVTMTRDTSTSTVYMELSSLRSEDTAVYYCAR |
| | | ERPLYAMDVWGQGTTVTVSS |
| NO: 6 | AB-3 | QVQLVQSGAEVKKPGASVKVSCKASGYTFTSYWMHWVRQAPGQGLEWM |
| | | GEIDPSGGSTNYNQKFQSRVTMTRDTSTSTVYMELSSLRSEDTAVYYCARE |
| | | RPLYASDVWGQGTTVTVSS |
| NO: 7 | AB-4 | QVQLVQSGAEVKKPGASVKVSCKASGYTFTSYYMHWVRQAPGQGLEWM |
| | | GEIRPSGGSTNYNEKFKSRVTMTRDTSTSTVYMELSSLRSEDTAVYYCARD |
| | | RPLYSMDVWGQGTTVTVSS |
| NO: 8 | AB-5 | QVQLVQSGAEVKKPGASVKVSCKASGYTFTSYWMHWVRQAPGQGLEWM |
| | | GEISPSGGSTNYNEKFKSRVTMTRDTSTSTVYMELSSLRSEDTAVYYCARE |
| | | RPLYAMDVWGQGTTVTVSS |
| NO: 9 | AB-6 | QVQLVQSGAEVKKPGASVKVSCKASGYTFTSYYMHWVRQAPGQGLEWM |
| | | GEISPFGGTTNYNEKFKSRVTMTRDTSTSTVYMELSSLRSEDTAVYYCARE |
| | | RPLYSSDVWGQGTTVTVSS |
| NO: 10 | AB-7 | QVQLVQSGAEVKKPGASVKVSCKASGYTFTSYYMHWVRQAPGQGLEWM |
| | | GEIFPSGGRTNYNEKFKSRVTMTRDTSTSTVYMELSSLRSEDTAVYYCARE |
| | | RPFYASDLWGQGTTVTVSS |
| NO: 11 | AB-8 | QVQLVQSGAEVKKPGASVKVSCKASGYTFTSYYMHWVRQAPGQGLEWM |
| | | GEILPSGGRTNYNEKFKSRVTMTRDTSTSTVYMELSSLRSEDTAVYYCARE |
| | | RPLYAMDIWGQGTTVTVSS |
| NO: 12 | AB-9 | QVQLVQSGAEVKKPGASVKVSCKASGYTFTSYYMHWVRQAPGQGLEWM |
| | | GEIKPSGGTSNYNEKFKSRVTMTRDTSTSTVYMELSSLRSEDTAVYYCARD |
| | | RPLYYSDVWGQGTTVTVSS |
| NO: 13 | AB-10 | QVQLVQSGAEVKKPGASVKVSCKASGYTFTSYYMHWVRQAPGQGLEWM |
| | | GEILPSGGRTNYNEKFKSRVTMTRDTSTSTVYMELSSLRSEDTAVYYCARE |
| | | RPLYAMDVWGQGTTVTVSS |
| NO: 14 | AB-11 | QVQLVQSGAEVKKPGASVKVSCKASGYSFTSYYMHWVRQAPGQGLEWM |
| | | GEIDLSGGSTNYNQKFQSRVTMTRDTSTSTVYMELSSLRSEDTAVYYCARE |
| | | RPLYAFDVWGQGTTVTVSS |

TABLE 2
VL Amino Acid Sequences

| SEQ ID | | Amino Acid Sequence |
|---|---|---|
| NO: 15 | Consensus | DIQMTQSPSSLSASVGDRVTITCRASQGISX₁₇X₁₈LAWYQQKPGKAPKLLIYSA |
| | | SX₁₉X₂₀YX₂₁GVPSRFSGSGSGTDFTFTISSLQPEDIATYYCQQX₂₂YSX₂₃X₂₄X₂₅T |
| | | FGQGTKLEIK |
| NO: 16 | Reference, | DIQMTQSPSSLSASVGDRVTITCRASQGISSALAWYQQKPGKAPKLLIYSASY |
| | AB-3, 5, 11 | RYTGVPSRFSGSGSGTDFTFTISSLQPEDIATYYCQQRYSLWRTFGQGTKLEIK |
| NO: 17 | AB-1 | DIQMTQSPSSLSASVGDRVTITCRASQGISTALAWYQQKPGKAPKLLIYSASY |
| | | LYTGVPSRFSGSGSGTDFTFTISSLQPEDIATYYCQQRYSLWRTFGQGTKLEIK |
| NO: 18 | AB-2 | DIQMTQSPSSLSASVGDRVTITCRASQGISGALAWYQQKPGKAPKLLIYSASY |
| | | RYTGVPSRFSGSGSGTDFTFTISSLQPEDIATYYCQQRYSLSRTFGQGTKLEIK |
| NO: 19 | AB-4 | DIQMTQSPSSLSASVGDRVTITCRASQGISSALAWYQQKPGKAPKLLIYSASY |
| | | RYTGVPSRFSGSGSGTDFTFTISSLQPEDIATYYCQQHYSSSWTFGQGTKLEIK |
| NO: 20 | AB-6 | DIQMTQSPSSLSASVGDRVTITCRASQGISSALAWYQQKPGKAPKLLIYSASY |
| | | LYSGVPSRFSGSGSGTDFTFTISSLQPEDIATYYCQQRYSLWYTFGQGTKLEIK |
| NO: 21 | AB-7 | DIQMTQSPSSLSASVGDRVTITCRASQGISSALAWYQQKPGKAPKLLIYSASN |
| | | LYTGVPSRFSGSGSGTDFTFTISSLQPEDIATYYCQQRYSLWRTFGQGTKLEIK |
| NO: 22 | AB-8 | DIQMTQSPSSLSASVGDRVTITCRASQGISSALAWYQQKPGKAPKLLIYSASY |
| | | RYTGVPSRFSGSGSGTDFTFTISSLQPEDIATYYCQQRYSTWRTFGQGTKLEIK |
| NO: 23 | AB-9 | DIQMTQSPSSLSASVGDRVTITCRASQGISGSLAWYQQKPGKAPKLLIYSASY |
| | | RYTGVPSRFSGSGSGTDFTFTISSLQPEDIATYYCQQYYSNYWTFGQGTKLEIK |
| NO: 24 | AB-10 | DIQMTQSPSSLSASVGDRVTITCRASQGISSALAWYQQKPGKAPKLLIYSASY |
| | | RYTGVPSRFSGSGSGTDFTFTISSLQPEDIATYYCQQRYSVALTFGQGTKLEIK |

TABLE 3
Amino Acid Sequences of CDRs

| | |
|---|---|
| SEQ ID | Amino Acid Sequence |

HCDR1

| | | |
|---|---|---|
| NO: 25 | Consensus | GYTFTSYX₁ |
| NO: 26 | Reference, AB-1, 2, 4, 6-10 | GYTFTSYY |
| NO: 27 | AB-3, 5 | GYTFTSYW |
| NO: 28 | AB-11 | GYSFTSYY |

HCDR2

| | | |
|---|---|---|
| NO: 29 | Consensus | IX₃PX₄GGX₅X₆ |
| NO: 30 | Reference | ISPFGGRT |
| NO: 31 | AB-1 | ILPGGGTT |
| NO: 32 | AB-2 | IEPFGGRT |
| NO: 33 | AB-3 | IDPSGGST |
| NO: 34 | AB-4 | IRPSGGST |
| NO: 35 | AB-5 | ISPSGGST |
| NO: 36 | AB-6 | ISPFGGTT |
| NO: 37 | AB-7 | IFPSGGRT |
| NO: 38 | AB-8, 10 | ILPSGGRT |
| NO: 39 | AB-9 | IKPSGGTS |
| NO: 40 | AB-11 | IDLSGGST |

HCDR3

| | | |
|---|---|---|
| NO: 41 | Consensus | ARX₉X₁₀PX₁₁YX₁₂X₁₃DX₁₄ |
| NO: 42 | Reference | ARERPLY ASDL |
| NO: 43 | AB-1 | ARESPLYASDI |
| NO: 44 | AB-2, 5, 10 | ARERPLYAMDV |
| NO: 45 | AB-3 | ARERPLYASDV |
| NO: 46 | AB-4 | ARDRPLYSMDV |
| NO: 47 | AB-6 | ARERPLYSSDV |
| NO: 48 | AB-7 | ARERPFYASDL |
| NO: 49 | AB-8 | ARERPLYAMDI |
| NO: 50 | AB-9 | ARDRPLYYSDV |
| NO: 51 | AB-11 | ARERPLYAFDV |

LCDR1

| | | |
|---|---|---|
| NO: 52 | Consensus | QGISX₁₅X₁₆ |
| NO: 53 | Reference, AB-3-8, 10, 11 | QGISSA |
| NO: 54 | AB-1 | QGISTA |
| NO: 69 | AB-2 | QGISGA |
| NO: 55 | AB-9 | QGISGS |

LCDR2

| | |
|---|---|
| Reference, AB-1-11 | SAS |

LCDR3

| | | |
|---|---|---|
| NO: 57 | Consensus | QQX₂₀YSX₂₁X₂₂X₂₃T |
| NO: 58 | Reference, AB-1, 3, 5, 7, 11 | QQRYSLWRT |
| NO: 59 | AB-2 | QQRYSLSRT |
| NO: 60 | AB-4 | QQHYSSSWT |
| NO: 61 | AB-6 | QQRYSLWYT |
| NO: 62 | AB-8 | QQRYSTWRT |
| NO: 63 | AB-9 | QQYYSNYWT |
| NO: 64 | AB-10 | QQRYSVALT |

TABLE 4
PCSK9 Binding Polypeptides

| | Number of | Relative Binding | | |
|---|---|---|---|---|
| | Amino Acid | to PCSK9 | | |
| | Mutational | Measured by | | |
| | Away from | DELIFA | | |
| Protein | Reference | Immunoassay | Functional | IC₅₀ (nM) |
| Reference | 0 | 6.6 | Yes | 19.97 |
| AB-1 | 8 | 1.8 | Yes | 18.37 |
| AB-2 | 6 | 3.1 | Yes | 17.19 |
| AB-3 | 7 | 2.8 | Yes | 16.06 |
| AB-4 | 11 | 2.9 | Yes | >53 |
| AB-5 | 5 | 4.3 | Yes | 18.85 |
| AB-6 | 6 | 1.8 | Yes | 16.51 |
| AB-7 | 5 | 2.0 | Yes | 18.11 |
| AB-8 | 5 | 2.9 | Yes | 18.35 |
| AB-9 | 13 | 3.9 | Yes | >53 |
| AB-10 | 7 | 4.3 | Yes | 20.15 |
| AB-11 | 9 | 2.4 | Yes | 20.79 |
| Control | N/A | 5.2 | Not | Not |
| | | | Determined | Determined |

TABLE 5
V_{H} (118 amino acids) Allowable Residues

| V_{H} position | Allowable | Not Allowable |
|---|---|---|
| 28 | T | S |
| 33 | W, Y | |
| 50 | E, W | |
| 52 | S, D, E, F, K, L, R | W, N, Y |
| 53 | P | L |
| 54 | F, G, S | E, T |
| 57 | R, S, T | H, I |
| 58 | S, T | |
| 62 | E, Q | |
| 65 | Q, K | |
| 99 | D, E | W |
| 100 | R, S | |
| 102 | L, F | |
| 103 | Y | F |
| 104 | A, S, Y | W |
| 105 | M, S | L |
| 107 | I, L, V | |

TABLE 6
V_{L} (107 amino acids) Allowable Residues

| V_{L} position | Allowable | Not Allowable |
|---|---|---|
| 28 | G | S |
| 31 | S, G, T | |
| 32 | S, A | |
| 53 | Y, N | |
| 54 | R, L | |
| 56 | S, T | |
| 91 | R, H, Y | V |
| 92 | Y | W |
| 93 | S | L |
| 94 | L, N, S, T, V | |
| 95 | W, A, S, Y | |
| 96 | R, L, W, Y | |

1. A polypeptide that specifically binds a proprotein convertase subtilisin/kexin type 9 (PCSK9), comprising:
a) an immunoglobulin heavy chain variable region (V_{H}) comprising an amino acid sequence that is at least 95% identical to SEQ ID NO:3; and
b) an immunoglobulin light chain variable region (V_{L}) comprising an amino acid sequence that is at least 95% identical to SEQ ID NO: 16.

2. (canceled)

3. The polypeptide of claim 1, wherein the polypeptide comprises a paratope that is identical to the paratope of an antibody comprising a V_{H} and a V_{L}, wherein the V_{H} and V_{L} of the antibody are selected from the following V_{H}/V_{L} combinations:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); and
SEQ ID NO:14/SEQ ID NO:16 (AB-11).

4. (canceled)

5. The polypeptide of claim 1, comprising the HCDR1, HCDR2 and HCDR3, and LCDR1, LCDR2 and LCDR3, of an antibody comprising a V_{H} and a V_{L}, wherein the V_{H} and V_{L} of the antibody are selected from the following V_{H}/V_{L} combinations:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); and
SEQ ID NO:14/SEQ ID NO:16 (AB-11).

6.-19. (canceled)

20. The polypeptide of claim 1, wherein the polypeptide comprises a V_{H}/V_{L} combination that is identical to the following V_{H}/V_{L} combination:
SEQ ID NO:4/SEQ ID NO:17 (AB-1);
SEQ ID NO:5/SEQ ID NO:18 (AB-2);
SEQ ID NO:6/SEQ ID NO:16 (AB-3);
SEQ ID NO:7/SEQ ID NO:19 (AB-4);
SEQ ID NO:8/SEQ ID NO:16 (AB-5);
SEQ ID NO:9/SEQ ID NO:20 (AB-6);
SEQ ID NO:10/SEQ ID NO:21 (AB-7);
SEQ ID NO:11/SEQ ID NO:22 (AB-8);
SEQ ID NO:12/SEQ ID NO:23 (AB-9);
SEQ ID NO:13/SEQ ID NO:24 (AB-10); and
SEQ ID NO:14/SEQ ID NO:16 (AB-11).

21. The polypeptide of claim 1, wherein the V_{H}, V_{L}, or both contain human framework regions.

22. The polypeptide of claim 1, wherein the polypeptide is an antibody or an antigen-binding fragment thereof.

23. The polypeptide of claim 22, wherein the antigen binding fragment is selected from an Fab, an F(ab′)₂, an Fab′, an scFv, or an Fv.

24. (canceled)

25. The polypeptide of claim 22, further comprising an antibody heavy chain constant region sequence, an antibody light chain constant region sequence, or both.

26. The polypeptide of claim 25, wherein the antibody heavy chain constant region is an IgA constant region, an IgD constant region, an IgE constant region, an IgG constant region or an IgM constant region.

27.-28. (canceled)

29. The polypeptide of claim 25, further comprising an antibody light chain constant region selected from the group consisting of a κ constant region or a λ constant region.

30. (canceled)

31. The polypeptide of claim 1, wherein the polypeptide is conjugated to a heterologous moiety.

32.-38. (canceled)

39. The polypeptide of claim 1, wherein the polypeptide:
a) binds a PCSK9 with an affinity of 1 μM or less;
b) competes with Reference Antibody for binding to PCSK9;
c) has a weaker self-association than Reference Antibody;
d) has a lower serum clearance rate than Reference Antibody in a subject (e.g., a human);
e) has a longer plasma half-life than Reference Antibody in a subject (e.g., a human); or
any combination of the foregoing.

40.-44. (canceled)

45. A fusion protein comprising the polypeptide of claim 1.

46. A polynucleotide comprising a sequence encoding the polypeptide of claim 1.

47. (canceled)

48. An expression vector comprising the polynucleotide of claim 46.

49. A host cell comprising the expression vector of claim 48.

50. A composition comprising the polypeptide of claim 1.

51. (canceled)

52. A method of modulating PCSK9 signaling in a mammalian cell, comprising contacting the cell with the composition of claim 50.

53. A method of treating a subject in need thereof, comprising administering an effective amount of the composition of claim 50 to the subject.

54. A method of treating or managing a disease or condition associated with an increased LDL level in the blood in a subject in need thereof, comprising administering an effective amount of the composition of claim 50 to the subject.

55.-70. (canceled)
